(19) United States
(12) Patent Application Publication
(10) Pub. No.: US 2014/0194388 A1
(43) Pub. Date: Jul. 10, 2014

(54) COMBINATION THERAPY OF HSP90 INHIBITORY COMPOUNDS WITH CHK INHIBITORS

(71) Applicants: David Proia, Newton, MA (US); Suqin He, West Roxbury, MA (US)

(72) Inventors: David Proia, Newton, MA (US); Suqin He, West Roxbury, MA (US)

(73) Assignee: SYNTA PHARMACEUTICALS CORP., Lexington, MA (US)

(21) Appl. No.: 14/119,396

(22) PCT Filed: May 25, 2012
(86) PCT No.: PCT/US2012/039519

§ 371 Date: Feb. 24, 2014

Related U.S. Application Data
(60) Provisional application No. 61/490,110, filed on May 26, 2011.

(51) Int. Cl. A61K 31/675; A61K 31/404; A61K 31/4535; A61K 31/551; A61K 31/517; A61K 31/519; A61K 31/4196; A61K 31/497

(52) U.S. Cl. CPC A61K 31/675; A61K 31/4196; A61K 31/404; A61K 31/497; A61K 31/551; A61K 31/517; A61K 31/519; A61K 31/4535
USPC 514/80; 514/384; 514/383; 514/419; 514/255.05; 514/220; 514/266.2; 514/259.31; 514/326

(57) ABSTRACT

A pharmaceutical composition comprising a CHK inhibitor, and an Hsp90 inhibitor according to the following formulae or tautomers, or pharmaceutically acceptable salts thereof, wherein the variables in the structural formulae are defined herein. Also provided are methods for treating a proliferative disorder in a subject in need thereof, using pharmaceutical compositions described herein.

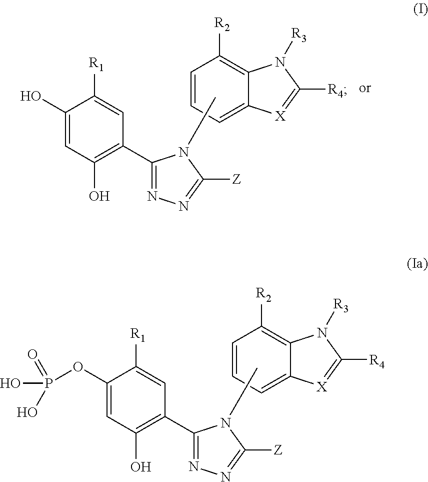

### CROSS-REFERENCE TO RELATED PATENTS

This application claims the benefit of priority to U.S. Provisional Patent Application No. 61/490,110, filed on May 26, 2011, the contents of which are incorporated herein by reference.

### BACKGROUND OF THE INVENTION

Although tremendous advances have been made in elucidating the genomic abnormalities that cause malignant cancer cells, currently available chemotherapy remains unsatisfactory, and the prognosis for the majority of patients diagnosed with cancer remains dismal. Most chemotherapeutic agents act on a specific molecular target thought to be involved in the development of the malignant phenotype. However, a complex network of signaling pathways regulate cell proliferation and the majority of malignant cancers are facilitated by multiple genetic abnormalities in these pathways. Therefore, it is less likely that a therapeutic agent that acts on one molecular target will be fully effective in curing a patient who has cancer.

Heat shock proteins (HSPs) are a class of chaperone proteins that are up-regulated in response to elevated temperature and other environmental stresses, such as ultraviolet light, nutrient deprivation and oxygen deprivation. HSPs act as chaperones to other cellular proteins (called client proteins), facilitate their proper folding and repair and aid in the refolding of misfolded client proteins. There are several known families of HSPs, each having its own set of client proteins. The Hsp90 family is one of the most abundant HSP families, accounting for about 1-2% of proteins in a cell that is not under stress and increasing to about 4-6% in a cell under stress. Inhibition of Hsp90 results in the degradation of its client proteins via the ubiquitin proteasome pathway. Unlike other chaperone proteins, the client proteins of Hsp90 are mostly protein kinases or transcription factors involved in signal transduction, and a number of its client proteins have been shown to be involved in the progression of cancer.

### SUMMARY OF THE INVENTION

It is now found that certain triazolone Hsp90 inhibitors and CHK inhibitor combinations are surprisingly effective at treating subjects with certain cancers without further increasing the side effect profile of the single agents. The particular combination therapies disclosed herein demonstrate surprising biological activity by demonstrating significant anticancer effects.

The present method utilizes Hsp90 inhibitors according to formulae (I) or (Ia), or at least one compound from Table 1 or 2 for the treatment of proliferative disorders, such as cancer, in combination with a CHK inhibitor. A method of treating a subject with cancer includes administering to the subject an Hsp90 inhibitor according to formulae (I) or (Ia), or at least one compound from Table 1 or 2 and a CHK inhibitor useful for the treatment of cancer. In one embodiment, the administration of the Hsp90 inhibitor and the CHK inhibitor are done concurrently. In another embodiment, the administration of the Hsp90 inhibitor and the CHK inhibitor are done sequentially. In another embodiment, the administration of the Hsp90 inhibitor and the CHK inhibitor are dosed independently. In any one of these embodiments, the CHK inhibitor may be 5-(3-fluorophenyl)-3-ureidothiophene-N—[(S)-piperidin-3-yl]-2-carboxamide (AZD7762), 7-nitro-1H-indole-2-carboxylic acid {4-[1-(guanidinohydrazone)-ethyl]-phenyl}-amide (PV1019), 5-[(8-chloro-3-isoquinolinyl)amino]-3-[(1R)-2-(dimethylamino)-1-methylethoxy]-2-pyrazinecarbonitrile (SAR-020106), PF-00477736, CCT241533, or SCH900776. In any one of these embodiments, the Hsp90 inhibitor may be a compound represented by formulae (I) or (Ia) or a compound in Tables 1 or 2.

In one embodiment, the method provides a kit for administration of the combination therapy having separate pharmaceutical compositions containing the Hsp90 inhibitor according to formulae (I) or (Ia), or at least one compound from Table 1 or 2, and the CHK inhibitor. In another embodiment, the kit includes one pharmaceutical composition containing both the Hsp90 inhibitor and the CHK inhibitor in the same composition. In any of these embodiments, each pharmaceutical composition may include one or more pharmaceutically acceptable carrier or diluent. In any one of these embodiments, the CHK inhibitor may be 5-(3-fluorophenyl)-3-ureidothiophene-N—[(S)-piperidin-3-yl]-2-carboxamide, 7-nitro-1H-indole-2-carboxylic acid {4-[1-(guanidinohydrazone)-ethyl]-phenyl}-amide, 5-[(8-chloro-3-isoquinolinyl)amino]-3-[(1R)-2-(dimethylamino)-1-methylethoxy]-2-pyrazinecarbonitrile, PF-00477736, CCT241533, or SCH900776. In any one of these embodiments, the Hsp90 inhibitor may be a compound represented in Tables 1 or 2.

In one embodiment, the method includes use of an Hsp90 inhibitor according to formulae (I) or (Ia) or at least one compound from Table 1 or 2 for the manufacture of a medicament for treating cancer in combination with a CHK inhibitor. In one embodiment, the cancer is non-small cell lung cancer. In one embodiment, the non-small cell lung cancer has a KRAS mutation. In one embodiment, the non-small cell lung cancer is ALK positive. In one embodiment, the non-small cell lung cancer has an EGFR mutation. In one embodiment, the non-small cell lung cancer has a BRAF mutation. In one embodiment, the cancer is melanoma. In one embodiment, the melanoma has a BRAF mutation.

In certain embodiments, the treatments utilize an Hsp90 inhibitory compound according to formulae (I) or (Ia) or at least one compound from Table 1 or 2 with a CHK inhibitor to help to arrest, partially or fully, or reduce the development of multidrug resistant cancerous cells in a subject. In this embodiment, the combinations may allow a reduced efficacious amount of the CHK inhibitor given to a subject, because the Hsp90 inhibitor should inhibit the development of multidrug-resistant cancerous cells. In one embodiment, the CHK inhibitor may be 5-(3-fluorophenyl)-3-ureidothiophene-N—[(S)-piperidin-3-yl]-2-carboxamide, 7-nitro-1H-indole-2-carboxylic acid {4-[1-(guanidinohydrazone)-ethyl]-phenyl}-amide, 5-[(8-chloro-3-isoquinolinyl)amino]-3-[(1R)-2-(dimethylamino)-1-methylethoxy]-2-pyrazinecarbonitrile, PF-00477736, CCT241533, or SCH900776. In another embodiment, the CHK inhibitor may be AZD7762.

### BRIEF DESCRIPTION OF THE DRAWINGS

The foregoing and other objects, features and advantages of the invention will be apparent from the following more particular description of some embodiments of the invention, as illustrated in the accompanying drawings in which like reference characters refer to the same parts throughout the different views. The drawings are not necessarily to scale, emphasis instead being placed upon illustrating the principles of the invention.

FIG. 1 shows ganetespib destabilizes the master cell cycle regulator CDK1 and the DNA damage checkpoint CHK1.

FIG. 2 shows synergistic inhibition of CHK signaling with AZD-7762 and ganetespib in killing PC3 cells.

### DETAILED DESCRIPTION OF THE INVENTION

### Definitions

Unless otherwise specified, the below terms used herein are defined as follows:

As used herein, the term “alkyl” means a saturated or unsaturated, straight chain or branched, non-cyclic hydrocarbon having from 1 to 10 carbon atoms. Representative straight chain alkyls include methyl, ethyl, n-propyl, n-butyl, n-pentyl, n-hexyl, n-heptyl, n-octyl, n-nonyl and n-decyl; while representative branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl, and the like. The term “(C₁-C₆)alkyl” means a saturated, straight chain or branched, non-cyclic hydrocarbon having from 1 to 6 carbon atoms. Alkyl groups included in compounds described herein may be optionally substituted with one or more substituents. Examples of unsaturated alkyls include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl, 3-decenyl, acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl, and the like. Alkyl groups included in compounds described herein may be optionally substituted with one or more substituents.

As used herein, the term “cycloalkyl” means a saturated or unsaturated, mono- or polycyclic, non-aromatic hydrocarbon having from 3 to 20 carbon atoms. Representative cycloalkyls include cyclopropyl, 1-methylcyclopropyl, cyclobutyl, cyclopentyl, cyclohexyl, cycloheptyl, cyclooctyl, cyclononyl, cyclodecyl, octahydropentalenyl, cyclohexenyl, cyclooctenyl, cyclohexynyl, and the like. Cycloalkyl groups included in compounds described herein may be optionally substituted with one or more substituents.

As used herein, the term “alkylene” refers to an alkyl group that has two points of attachment. The term “(C₁-C₆)alkylene” refers to an alkylene group that has from one to six carbon atoms. Straight chain (C₁-C₆)alkylene groups are preferred. Non-limiting examples of alkylene groups include methylene (—CH₂—), ethylene (—CH₂CH₂—), n-propylene (—CH₂CH₂CH₂—), isopropylene (—CH₂CH(CH₃)—), and the like. Alkylene groups may be saturated or unsaturated, and may be optionally substituted with one or more substituents.

As used herein, the term “lower” refers to a group having up to four atoms. For example, a “lower alkyl” refers to an alkyl radical having from 1 to 4 carbon atoms, “lower alkoxy” refers to “—O—(C₁-C₄)alkyl.

As used herein, the term “haloalkyl” means an alkyl group, in which one or more, including all, the hydrogen radicals are replaced by a halo group(s), wherein each halo group is independently selected from —F, —Cl, —Br, and —I. For example, the term “halomethyl” means a methyl in which one to three hydrogen radical(s) have been replaced by a halo group. Representative haloalkyl groups include trifluoromethyl, bromomethyl, 1,2-dichloroethyl, 4-iodobutyl, 2-fluoropentyl, and the like.

As used herein, an “alkoxy” is an alkyl group which is attached to another moiety via an oxygen linker. Alkoxy groups included in compounds described herein may be optionally substituted with one or more substituents.

As used herein, a “haloalkoxy” is a haloalkyl group which is attached to another moiety via an oxygen linker.

As used herein, the term an “aromatic ring” or “aryl” means a mono- or polycyclic hydrocarbon, containing from 6 to 15 carbon atoms, in which at least one ring is aromatic. Examples of suitable aryl groups include phenyl, tolyl, anthracenyl, fluorenyl, indenyl, azulenyl, and naphthyl, as well as benzo-fused carbocyclic moieties such as 5,6,7,8-tetrahydronaphthyl. Aryl groups included in compounds described herein may be optionally substituted with one or more substituents. In one embodiment, the aryl group is a monocyclic ring, wherein the ring comprises 6 carbon atoms, referred to herein as “(C₆)aryl.”

As used herein, the term “aralkyl” means an aryl group that is attached to another group by a (C₁-C₆)alkylene group. Representative aralkyl groups include benzyl, 2-phenyl-ethyl, naphth-3-yl-methyl and the like. Aralkyl groups included in compounds described herein may be optionally substituted with one or more substituents.

As used herein, the term “heterocyclyl” means a monocyclic or a polycyclic, saturated or unsaturated, non-aromatic ring or ring system which typically contains 5- to 20-members and at least one heteroatom. A heterocyclic ring system can contain saturated ring(s) or unsaturated non-aromatic ring(s), or a mixture thereof. A 3- to 10-membered heterocycle can contain up to 5 heteroatoms, and a 7- to 20-membered heterocycle can contain up to 7 heteroatoms. Typically, a heterocycle has at least one carbon atom ring member. Each heteroatom is independently selected from nitrogen, which can be oxidized (e.g., N(O)) or quaternized, oxygen and sulfur, including sulfoxide and sulfone. The heterocycle may be attached via any heteroatom or carbon atom. Representative heterocycles include morpholinyl, thiomorpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyrindinyl, tetrahydropyrimidinyl, tetrahydrothiophenyl, tetrahydrothiopyranyl, and the like. A heteroatom may be substituted with a protecting group known to those of ordinary skill in the art, for example, a nitrogen atom may be substituted with a tert-butoxycarbonyl group. Furthermore, the heterocyclyl included in compounds described herein may be optionally substituted with one or more substituents. Only stable isomers of such substituted heterocyclic groups are contemplated in this definition.

As used herein, the term “heteroaryl”, or like terms, means a monocyclic or a polycyclic, unsaturated radical containing at least one heteroatom, in which at least one ring is aromatic. Polycyclic heteroaryl rings must contain at least one heteroatom, but not all rings of a polycyclic heteroaryl moiety must contain heteroatoms. Each heteroatom is independently selected from nitrogen, which can be oxidized (e.g., N(O)) or quaternized, oxygen and sulfur, including sulfoxide and sulfone. Representative heteroaryl groups include pyridyl, 1-oxo-pyridyl, furanyl, benzo[1,3]dioxolyl, benzo[1,4]dioxinyl, thienyl, pyrrolyl, oxazolyl, imidazolyl, thiazolyl, an isoxazolyl, quinolinyl, pyrazolyl, isothiazolyl, pyridazinyl, pyrimidinyl, pyrazinyl, a triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzothienyl. In one embodiment, the heteroaromatic ring is selected from 5-8 membered monocyclic heteroaryl rings. The point of attachment of a heteroaromatic or heteroaryl ring may be at either a carbon atom or a heteroatom. Heteroaryl groups included in compounds described herein may be optionally substituted with one or more substituents. As used herein, the term “(C₅)heteroaryl” means an heteroaromatic ring of 5 members, wherein at least one carbon atom of the ring is replaced with a heteroatom, such as oxygen, sulfur or nitrogen. Representative (C₅)heteroaryls include furanyl, thienyl, pyrrolyl, oxazolyl, imidazolyl, thiazolyl, isoxazolyl, pyrazolyl, isothiazolyl, pyrazinyl, triazolyl, thiadiazolyl, and the like. As used herein, the term “(C₆)heteroaryl” means an aromatic heterocyclic ring of 6 members, wherein at least one carbon atom of the ring is replaced with a heteroatom such as oxygen, nitrogen or sulfur. Representative (C₆)heteroaryls include pyridyl, pyridazinyl, pyrazinyl, triazinyl, tetrazinyl, and the like.

As used herein, the term “heteroaralkyl” means a heteroaryl group that is attached to another group by a (C₁-C₆)alkylene. Representative heteroaralkyls include 2-(pyridin-4-yl)-propyl, 2-(thien-3-yl)-ethyl, imidazol-4-yl-methyl, and the like. Heteroaralkyl groups included in compounds described herein may be optionally substituted with one or more substituents.

As used herein, the term “halogen” or “halo” means —F, —Cl, —Br or —I.

Suitable substituents for an alkyl, alkylene, alkenyl, alkynyl, cycloalkyl, cycloalkenyl, heterocyclyl, aryl, aralkyl, heteroaryl, and heteroaralkyl groups include are those substituents which form a stable compound described herein without significantly adversely affecting the reactivity or biological activity of the compound described herein. Examples of substituents for an alkyl, alkylene, alkenyl, alkynyl, cycloalkyl, cycloalkenyl, heterocyclyl, aryl, aralkyl, heteroaryl, and heteroaralkyl include an alkyl, alkenyl, alkynyl, cycloalkyl, cycloalkenyl, heterocyclyl, aryl, heteroaryl, aralkyl, heteraralkyl, heteroalkyl, alkoxy, (each of which can be optionally and independently substituted), —C(O)NR²⁸R²⁹, —C(S)NR²⁸R²⁹, —C(NR³²)NR²⁸R²⁹, NR³³C(O)R³¹, —NR³³C(S)R³¹, —NR³³C(NR³²)R³¹, halo, —OR³³, cyano, nitro, —C(O)R³³, —C(S)R³³, —C(NR³²)R³³, —NR²⁸R²⁹, —C(O)OR³³, —C(S)OR³³, —C(NR³²)OR³³, —OC(O)R³³, —OC(S)R³³, —OC(NR³²)R³³, —NR³⁰C(O)NR²⁸R²⁹, —NR³³C(S)NR²⁸R²⁹, —NR³³C(NR³²)NR²⁸R²⁹, —OC(O)NR²⁸R²⁹, —OC(S)NR²⁸R²⁹, —OC(NR³²)NR²⁸R²⁹, —NR³³C(O)OR³¹, —NR³³C(S)OR³¹, —NR³³C(NR³²)OR³¹, —S(O)ₖR³³, —OS(O)ₖR³³, —NR³³S(O)ₖR³³, —S(O)ₖNR²⁸R²⁹, —OS(O)ₖNR²⁸R²⁹, —NR³³S(O)ₖNR²⁸R²⁹, guanidino, —C(O)SR³¹, —C(S)SR″, —C(NR³²)SR³¹, —OC(O)OR³¹, —OC(S)OR³¹, —OC(NR³²)OR³¹, —SC(O)R³³, —SC(O)OR³¹, —SC(NR³²)OR³¹, —SC(S)R³³, —SC(S)OR³¹, —SC(O)NR²⁸R²⁹, —SC(NR³²)NR²⁸R²⁹, —SC(S)NR²⁸R²⁹, —SC(NR³²)R³³, —OS(O)ₖOR³¹, —S(O)ₖOR³¹, —NR³⁰S(O)ₖOR³¹, —SS(O)ₖR³³, —SS(O)ₖOR³¹, —SS(O)ₖNR²⁸R²⁹, —OP(O)(OR³¹)₂, or —SP(O)(OR³¹)₂. In addition, any saturated portion of an alkyl, cycloalkyl, alkylene, heterocyclyl, alkenyl, cycloalkenyl, alkynyl, aralkyl and heteroaralkyl groups, may also be substituted with ═O, ═S, or ═N—R³². Each R²⁸ and R²⁹ is independently H, alkyl, alkenyl, alkynyl, cycloalkyl, cycloalkenyl, heterocyclyl, aryl, heteroaryl, aralkyl, or heteraralkyl, wherein each alkyl, alkenyl, alkynyl, cycloalkyl, cycloalkenyl, heterocyclyl, aryl, heteroaryl, aralkyl, or heteroalkyl represented by R²⁸ or R²⁹ is optionally and independently substituted. Each R³⁰, R³¹ and R³³ is independently H, alkyl, alkenyl, alkynyl, cycloalkyl, cycloalkenyl, heterocyclyl, aryl, heteroaryl, aralkyl, or heteraralkyl, wherein each alkyl, alkenyl, alkynyl, cycloalkyl, cycloalkenyl, heterocyclyl, aryl, heteroaryl, aralkyl, and heteraralkyl represented by R³⁰ or R³¹ or R³³ is optionally and independently unsubstituted. Each R³² is independently H, alkyl, alkenyl, alkynyl, cycloalkyl, cycloalkenyl, heterocyclyl, aryl, heteroaryl, aralkyl, heteraralkyl, —C(O)R³³, —C(O)NR²⁸R²⁹, —S(O)ₖR³³, or —S(O)ₖNR²⁸R²⁹, wherein each alkyl, alkenyl, alkynyl, cycloalkyl, cycloalkenyl, heterocyclyl, aryl, heteroaryl, aralkyl and heteraralkyl represented by R³² is optionally and independently substituted. The variable k is 0, 1 or 2. In some embodiments, suitable substituents include C₁-C₄ alkyl, C₁-C₄ haloalkyl, C₁-C₄ alkoxy, C₁-C₄ haloalkoxy, C₁-C₄ hydroxyalkyl, halo, or hydroxyl.

When a heterocyclyl, heteroaryl or heteroaralkyl group contains a nitrogen atom, it may be substituted or unsubstituted. When a nitrogen atom in the aromatic ring of a heteroaryl group has a substituent, the nitrogen may be oxidized or a quaternary nitrogen.

As used herein, the terms “subject”, “patient” and “mammal” are used interchangeably. The terms “subject” and “patient” refer to an animal (e.g., a bird such as a chicken, quail or turkey, or a mammal), preferably a mammal including a non-primate (e.g., a cow, pig, horse, sheep, rabbit, guinea pig, rat, cat, dog, and mouse) and a primate (e.g., a monkey, chimpanzee and a human), and more preferably a human. In one embodiment, the subject is a non-human animal such as a farm animal (e.g., a horse, cow, pig or sheep), or a pet (e.g., a dog, cat, guinea pig or rabbit). In another embodiment, the subject is a human.

Unless indicated otherwise, the compounds described herein containing reactive functional groups, such as carboxy, hydroxy, thiol and amino moieties, also include corresponding protected derivatives thereof. “Protected derivatives” are those compounds in which a reactive site or sites are blocked with one ore more protecting groups. Examples of suitable protecting groups for hydroxyl groups include benzyl, methoxymethyl, allyl, trimethylsilyl, tert-butyldimethylsilyl, acetate, and the like. Examples of suitable amine protecting groups include benzyloxycarbonyl, tert-butoxycarbonyl, tert-butyl, benzyl and fluorenylmethyloxy-carbonyl (Fmoc). Examples of suitable thiol protecting groups include benzyl, tert-butyl, acetyl, methoxymethyl and the like. Other suitable protecting groups are well known to those of ordinary skill in the art and include those found in T. W. GREENE, PROTECTING GROUPS IN ORGANIC SYNTHESIS, (John Wiley & Sons, Inc., 1981).

As used herein, the term “compound(s) described herein” or similar terms refers to a compound of formulae (I), or (Ia) or at least one compound from Table 1 or 2 or a tautomer or pharmaceutically acceptable salt thereof. Also included in the scope of the embodiments are a solvate, clathrate, hydrate, polymorph, prodrug, or protected derivative of a compound of formulae (I), or (Ia), or at least one compound from Table 1 or 2.

The compounds described herein may contain one or more chiral centers and/or double bonds and, therefore, exist as stereoisomers, such as double-bond isomers (i.e., geometric isomers), enantiomers or diastereomers. Each chemical structure shown herein, including the compounds described herein, encompass all of the corresponding compound's enantiomers, diastereomers and geometric isomers, that is, both the stereochemically pure form (e.g., geometrically pure, enantiomerically pure, or diastereomerically pure) and isomeric mixtures (e.g., enantiomeric, diastereomeric and geometric isomeric mixtures). In some cases, one enantiomer, diastereomer or geometric isomer will possess superior activity or an improved toxicity or kinetic profile compared to other isomers. In those cases, such enantiomers, diastereomers and geometric isomers of compounds described herein are preferred.

When a disclosed compound is named or depicted by structure, it is to be understood that solvates (e.g., hydrates) of the compound or a pharmaceutically acceptable salt thereof is also included. “Solvates” refer to crystalline forms wherein solvent molecules are incorporated into the crystal lattice during crystallization. Solvates may include water or nonaqueous solvents such as ethanol, isopropanol, DMSO, acetic acid, ethanolamine and ethyl acetate. When water is the solvent molecule incorporated into the crystal lattice of a solvate, it is typically referred to as a “hydrate”. Hydrates include stoichiometric hydrates as well as compositions containing variable amounts of water.

When a disclosed compound is named or depicted by structure, it is to be understood that the compound, including solvates thereof, may exist in crystalline forms, non-crystalline forms or a mixture thereof. The compounds or solvates may also exhibit polymorphism (i.e., the capacity to occur in different crystalline forms). These different crystalline forms are typically known as “polymorphs.” It is to be understood that when named or depicted by structure, the disclosed compounds and solvates (e.g., hydrates) also include all polymorphs thereof. Polymorphs have the same chemical composition but differ in packing, geometrical arrangement and other descriptive properties of the crystalline solid state. Polymorphs, therefore, may have different physical properties such as shape, density, hardness, deformability, stability and dissolution properties. Polymorphs typically exhibit different melting points, IR spectra and X-ray powder diffraction patterns, which may be used for identification. One of ordinary skill in the art will appreciate that different polymorphs may be produced, for example, by changing or adjusting the conditions used in crystallizing the compound. For example, changes in temperature, pressure or solvent may result in different polymorphs. In addition, one polymorph may spontaneously convert to another polymorph under certain conditions.

When a disclosed compound is named or depicted by structure, it is to be understood that clathrates (“inclusion compounds”) of the compound or its pharmaceutically acceptable salt, solvate or polymorph, are also included. “Clathrate” means a compound described herein, or a salt thereof, in the form of a crystal lattice that contains spaces (e.g., channels) that have a guest molecule trapped within (e.g., a solvent or water).

As used herein, and unless otherwise indicated, the term “prodrug” means a derivative of a compound that can hydrolyze, oxidize, or otherwise react under biological conditions (in vitro or in vivo) to provide a compound described herein. Prodrugs may become active upon such reaction under biological conditions, or they may have activity in their unreacted forms. Examples of prodrugs contemplated herein include analogs or derivatives of compounds of formulae (I) or (Ia) or a compound in Tables 1 or 2 that comprise biohydrolyzable moieties such as biohydrolyzable amides, biohydrolyzable esters, biohydrolyzable carbamates, biohydrolyzable carbonates, biohydrolyzable ureides and phosphate analogues. Prodrugs can be prepared using well-known methods, such as those described by BURGER'S MEDICINAL CHEMISTRY AND DRUG DISCOVERY, (Manfred E. Wolff Ed., 5th ed. (1995)) 172-178, 949-982.

As used herein, “Hsp90” includes each member of the family of heat shock proteins having a mass of about 90-kiloDaltons. For example, in humans the highly conserved Hsp90 family includes the cytosolic Hsp90α and Hsp90β isoforms, as well as GRP94, which is found in the endoplasmic reticulum, and HSP75/TRAP1, which is found in the mitochondrial matrix.

The CHK inhibitors herein refer to checkpoint kinase inhibitors. The CHK inhibitors used herein include 5-(3-fluorophenyl)-3-ureidothiophene-N—[(S)-piperidin-3-yl]-2-carboxamide (a/k/a AZD7762), 7-nitro-1H-indole-2-carboxylic acid {4-[1-(guanidinohydrazone)-ethyl]-phenyl}-amide (a/k/a PV1019), 5-[(8-chloro-3-isoquinolinyl)amino]-3-[(1R)-2-(dimethylamino)-1-methylethoxy]-2-pyrazinecarbonitrile (a/k/a SAR-020106), PF-00477736, CCT241533, and SCH900776.

The KRAS oncogene (the cellular homolog of the Kirsten rat sarcoma virus gene, Accession No. NP_{—}203524) is a critical gene in the development of a variety of cancers, and the mutation status of this gene is an important characteristic of many cancers. Mutation status of the gene can provide diagnostic, prognostic and predictive information for several cancers. The KRAS gene is a member of a family of genes (KRAS, NRAS and HRAS). KRAS is a member of the RAS family of oncogenes, a collection of small guanosine triphosphate (GTP)-binding proteins that integrate extracellular cues and activate intracellular signaling pathways to regulate cell proliferation, differentiation, and survival. Gain-of-function mutations that confer transforming capacity are frequently observed in KRAS, predominantly arising as single amino acid substitutions at amino acid residues G12, G13 or Q61. Constitutive activation of KRAS leads to the persistent stimulation of downstream signaling pathways that promote tumorigenesis, including the RAF/MEK/ERK and PI3K/AKT/mTOR cascades. In NSCLC, KRAS mutations are highly prevalent (20-30%) and are associated with unfavorable clinical outcomes. Mutations in KRAS appear mutually exclusive with those in EGFR in NSCLC tumors; more importantly, they can account for primary resistance to targeted EGFR TKI therapies. Mutations in the KRAS gene are common in many types of cancer, including pancreatic cancer (˜65%), colon cancer (˜40%), lung cancer (˜20%) and ovarian cancer (˜15%).

A variety of laboratory methods have been utilized to detect mutations in the KRAS gene. See, e.g., Jimeno et al, KRAS mutations and sensitivity to epidermal growth factor receptor inhibitors in colorectal cancer: practical application of patient selection. J. Clin. Oncol. 27, 1130-1135 (2009); Van Krieken et al. KRAS mutation testing for predicting response to anti-EGFR therapy for colorectal carcinoma: proposal for a European quality assurance program. Virchows Archiv. 453, 417-431 (2008). Most methods include the use of PCR to amplify the appropriate region of the KRAS gene, including exons 2 and 3, and then utilize different methods to distinguish wild-type from mutant sequences in key codons, such as 12 and 13. The detection methods include nucleic acid sequencing, allele-specific PCR methods, single-strand conformational polymorphism analysis, melt-curve analysis, probe hybridization and others. The main features for consideration for these molecular techniques are the ability to distinguish the appropriate spectrum of variants at the codons of interest and the sensitivity or limit of detection (LOD) for mutant alleles. Both of these parameters are important, given the fact that tumors may be very heterogeneous, both with regard to the percentage of tumor cells within a given tissue and the potential for genetic heterogeneity.

More over, many methods have also been developed for KRAS mutation analysis to address various specific issues, related to increased analytical sensitivity, and they include allele-specific PCR using amplification refractory mutation system (ARMS) technology or co-amplification at a lower denaturation temperature-PCR methods, pyrosequencing approaches and real-time PCR methods that use specific probe technologies, such as peptide nucleic acids. See, e.g., Pritchard et al, COLD-PCR enhanced melting curve analysis improves diagnostic accuracy for KRAS mutations in colorectal carcinoma. BMC Clin. Pathol. 10, 1-10 (2010); Weichart et al, KRAS genotyping of paraffin-embedded colorectal cancer tissue in routine diagnostics: comparison of methods and impact of histology. J. Mol. Diagn. 12, 35-42 (2010); Oliner et al, A comparability study of 5 commercial KRAS tests. Diagn. Pathol. 5, 23-29 (2010); Ogino et al, Brahmandan M et al. Sensitive sequencing method for KRAS mutation detection by pyrosequencing. J. Mol. Diagn. 4, 413-421 (2005).

There are several examples of laboratory-developed tests (LDTs) for detecting KRAS mutations, as well as a series of kits for research and for use in clinical diagnostics. For example, the TheraScreen® assay (DxS, Manchester, UK) is a CE-marked kit intended for the detection and qualitative assessment of seven somatic mutations in the KRAS gene, to aid clinicians in the identification of colorectal cancer patients who may benefit from anti-EGFR therapies, such as panitumumab and cetuximab. This assay uses an amplification refractory mutation system (ARMS), which is a version of allele-specific PCR; and detection of amplification products with Scorpion™ probes. See, e.g., TheraScreen® Package Insert, DsX, Manchester, UK (2009); Whitehall et al, A multicenter blinded study to evaluate KRAS mutation testing methodologies in the clinical setting. J. Mol. Diagn. 11, 543-552 (2009); Oliner et al, A comparability study of 5 commercial KRAS tests. Diagn. Pathol. 5, 23-29 (2010).

In addition, the European Society of Pathology (ESP), to help evaluate the reliability of KRAS mutation testing, has established a quality assurance program for KRAS mutation analysis in colorectal cancers at http://kras.equascheme.org.

The ALK (anaplastic lymphoma kinase, Accession No. NP_{—}004295) RTK (receptor tyrosine kinase) was originally identified as a member of the insulin receptor subfamily of RTKs that acquires transforming capability when truncated and fused to NPM (nucleophosmin) in the t(2;5) chromosomal rearrangement associated with ALCL (anaplastic large cell lymphoma). To date, many chromosomal rearrangements leading to enhanced ALK activity have been described and are implicated in a number of cancer types. Recent reports of the EML4 (echinoderm microtubule-associated protein like 4)-ALK oncoprotein in NSCLC, together with the identification of activating point mutations in neuroblastoma, have highlighted ALK as a significant player and target for drug development in cancer. Representative ALK abnormalities (or “ALK+”) include EML4-ALK fusions, KIF5B-ALK fusions, TGF-ALK fusions, NPM-ALK fusions, and ALK point mutations.

The following two assays are presented for general information about detection and identification of ALK alterations, mutations or rearrangements in an ALK gene or gene product. These types of assays were also used in obtaining the results in Examples 1 and 2 herein.

The EML4/ALK assay detects eight known fusion variants and other undefined variants, in conjunction with measuring expression of wild type EML4 and ALK 5′ and 3′.

Lung cancer is the most common and deadly form of cancer in the USA, with a 5-year survival rate of approximately 15 percent. A subset of NSCLC patients have translocations which fuse the 5′ end of the EML4 gene to the 3′ end of the ALK gene creating an activated ALK oncogene. The incidence of ALK activation in NSCLC is low (2-7 percent), but it may be as high as 13 percent in patients with adenocarcinoma, no or a light history of smoking, younger age, and WT EGFR and KRAS genes. There are several other adenocarcinomas for which the ALK activation is relevant: breast, bladder, head & neck, and colon. Of particular interest, 5% of primary and metastatic melanoma patients harbor the translocation as well.

The EML4/ALK fusion protein displays constitutive ALK kinase activity, which can be targeted with ALK kinase inhibitors. The presence of an EML4/ALK translocation predicts a favorable response to ALK inhibitor therapy.

The quantitative Nuclease Protection Assay (qNPA™) is a multiplexed, lysis only assay of mRNA (53-58) that can also measure DNA and miRNA. What sets qNPA apart from other assays is that it does not require extraction of the DNA or RNA, but rather uses directly lysed samples. This permits high sample throughput, combined with the simultaneous measurement of DNA, mRNA and miRNA from the same lysate, and if necessary, on the same array.

qNPA also is very precise, with average whole assay CV's from tissues <10%, which means changes <1.2-fold can be detected, p<0.05. It is currently available as a low cost array plate-based assay measuring up to 47 genes/well.

Genetics: Multiple inversions on chromosome 2p generate in-frame fusions of the EML4 and ALK genes. While the breakpoints of EML4 can vary (fusion at exons 2, 6, 13, 14, 15, 18, and 20), the breakpoint of ALK occurs consistently at exon 20, 5′ of the kinase domain. The majority (˜70 percent) of translocations involve EML4 exon 13 (variant 1) or EML4 exon 6a/b (variant 3a/b). Due to close proximity of the EML4 and ALK genes, thus the small inversions, detection of some EML4/ALK variants is challenging with commercially available ALK break-apart FISH probes.

Product Format: The initial product is based upon the qNPA ArrayPlate format, either in 47 or 16 spot format as appropriate and dictated by the number of analytes to be tested with the ALK array.

Components: Kits are all inclusive with step-by-step instructions for ease of use.

Sample Type: Cell Lines, Blood, Purified RNA or FFPE

Intended Uses

The intended use for this product is to detect any of the specified expression wild types and fusion variants of ALK and EML4/ALK.

These are as follows:

WT: ALK-5′

WT: ALK-3′

Fusion: EML4/ALK-variant 1

Fusion: EML4/ALK-variant 2

Fusion: EML4/ALK-variant 3a

Fusion: EML4/ALK-variant 3b

Fusion: EML4/ALK-variant 4

Fusion: EML4/ALK-variant 5a

Fusion: EML4/ALK-variant 5b

Fusion: EML4/ALK-variant 6

Fusion: KIF5B-ALK

Fusion: TFG-ALK

WT: EML4-5′

WT: KIF5B-5′

WT: TFG-5″

Insight ALK Screen is an RT-qPCR assay that detects the presence of ALK fusions and upregulation of ALK wild type (which is abnormal in adult tissue outside the central nervous system and can be indicative of ALK-driven disease). The assay uses a three tube reaction series (plus controls) to measure expression of the extracellular segment of ALK (ALK WT), ALK kinase domain expression (ALK Kinase), and expression of an internal reference gene, Cytochrome c oxidase subunit 5B (COX5B). By focusing on relative expression of the ALK gene, Insight ALK Screen can more accurately detect the presence of ALK fusions than a variant-specific PCR approach that targets the 10+ unique 5′ gene partners, such as EML4.

Methods and procedures for the detection of wild type ALK and NPM-ALK fusions can be found in U.S. Pat. Nos. 5,529,925 and 5,770,421.

The Raf family of proto-oncogenes (A-raf, B-raf and C-raf) was first identified when C-raf was discovered due to its homology with v-raf, the transforming gene of the mouse sarcoma virus 3611. A-raf was later discovered by screening a cDNA library under low stringency conditions using a v-raf probe, and B-raf was discovered due to its homology with C-Rmil, a transforming gene in avian retrovirus Mill Hill No. 2. The Raf family of proteins is involved in the Ras/Raf/MEK/ERK pathway, referred to herein as the “MAP kinase pathway” (MEK stands for “MAPK/ERK kinase” and ERK stands for “extracellularly regulated kinases”), which has been implicated in the genesis and progression of many human cancers through upregulation of cell division and proliferation. All raf proteins are serine/threonine kinases which are capable of activating the MAP kinase pathway. However, B-raf is far more potent at activating this pathway than A-raf or C-raf, and mutations in the gene encoding B-raf are more common in cancer. For example, B-raf mutations have been identified in 60% to 70% of malignant melanomas, 83% of anaplastic thyroid carcinoma, 35% to 69% of papillary thyroid carcinoma, 4% to 16% of colon cancer, 63% of low-grade ovarian carcinoma, 15% of Barrett's esophageal carcinoma, 4% of acute myeloid leukemia, 3-4.8% of head and neck squamous cell carcinoma, 2%-3% of non-small-cell lung cancer, 2% of gastric carcinoma, 2% of non-Hodgkin's lymphoma and has been reported in glioma, sarcoma, breast cancer, cholangiocarcinoma, and liver cancer. Most mutations in B-raf that have been found in human cancers are point mutations that occur in the kinase domain and are clustered in exons 11 and 15 of the gene which contains several regulatory phosphorylation sites (S446, S447, D448, D449, T599, and S602). (Beeram, et al., Journal of Clinical Oncology (2005), 23(27):6771-6790). The most prevalent mutation is the T1799A transversion mutation which accounts for more than 80% of mutations in the BRAF gene and results in a V600E mutation in B-raf. The V600E was formerly designated V599E (the gene mutation was designated T1796A) due to a mistake in the GenBank nucleotide sequence NM 004333. The corrected GenBank sequence is NT 007914 and designates the protein mutation as V600E and the gene mutation as T1799A. This corrected numbering will be used herein. This mutation is thought to mimic phosphorylation in the activation segment of B-raf since it inserts a negatively charged residue near two activating phosphorylation sites, T599 and S602, and thus results in constitutively active B-raf in a Ras independent manner. (Xing, M., Endocrine-Related Cancer (2005), 12:245-262).

Treatment of cancer cells with 17AAG has been shown to stimulate the degradation of B-raf, and mutant forms of B-raf have been shown to be more sensitive to degradation than the wild type. For example, when melanoma cell line A375 which contain the V600E mutation was treated with 17AAG, B-raf was degraded more rapidly than in CHL cells which contained wild type B-raf. Other B-raf mutants (e.g., V600D, G469A, G469E, G596R, G466V, and G594V) were a found to be degraded more rapidly than wild type B-raf when transvected into COS cells. However, B-raf mutants E586K and L597V were not sensitive to degradation when cells were treated with 17AAG. Therefore, it is believed that wild type B-raf in its activated form is a client protein of Hsp90 and that most mutated forms of B-raf are more dependent on Hsp90 for folding, stability and/or function than the wild type protein. (Dias, et al., Cancer Res. (2005), 65(23): 10686-10691). The B-raf inhibitors as used herein include PLX-4032 (vemurafenib, CAS No.: 918504-65-1), GDC-0879 (CAS No.: 905281-76-7), PLX-4720 (CAS No.: 918505-84-7), and sorafenib (Nexavar®) (CAS No.: 475207-59-1).

As used herein, a “subject with a mutation” in KRAS, ALK, EGFR, BRAF or other gene associated with cancer, or a “subject with a cancer with a mutation” in KRAS, ALK, EGFR, BRAF or other gene associated with cancer, and the like, are understood as a subject having cancer, wherein the tumor has at least one alteration (e.g., 1, 2, 3, 4, 5, 6, 7, 8, 9, 10, or more) in the indicated gene from the wild-type sequence in the gene and/or transcriptional, translational, and/or splicing control regions of the gene that result in the cell becoming cancerous, e.g., developing characteristics such as uncontrolled proliferation, immortality, metastatic potential, rapid growth and proliferation rate, decreased cell death/apoptosis, and certain characteristic morphological features. Mutations include, insertions, deletions, truncations, point mutations, and translocations. Mutations within a gene product can result in constituent activation of the gene product. Mutations that include alterations in transcriptional, translational, or splicing control regions can result in aberrant expression, typically over-expression, of a wild-type gene product. It is understood that not all gene mutations, even in oncogenes, result in a cell becoming cancerous. Mutations that result in oncogenesis are well known in the art. Methods to test mutations for oncogenic activity are well known in the art.

A mutation can be detected using any of a number of known methods in the art. The specific method to detect the mutation will depend, for example, on the type of mutation to be detected. For example, alterations in nucleic acid sequences can be easily detected using polymerase chain reaction and fluorescence in situ hybridization methods (FISH). Protein expression levels can be detected, for example, using immunohistochemistry. An aberrant expression level of a wild-type protein can be used as a surrogate for detection of a mutation in a transcriptional, translational, and/or splicing control regions of the gene without direct detection of the specific genetic change in the nucleic acid in the subject sample. The specific method of detection of the mutation is not a limitation of the invention. Methods to compare protein expression levels to appropriate controls are well known in the art.

In a preferred embodiment, when multiple tests are used to detect a mutation and one is positive, the mutation is considered to be present. The methods do not require that multiple assays be performed to detect a mutation.

As used herein, and in the art, an “ALK+” tumor or cancer is understood as a tumor or cancer that has a mutation such that ALK is overexpressed and causes a cancerous phenotype in the cell.

As used herein, a subject with a “wild-type” KRAS, ALK, EGFR, BRAF or other gene associated with cancer, or a “subject with a cancer with a wild-type” KRAS, ALK, EGFR, BRAF or other gene associated with cancer, and the like, are understood as a subject suffering from cancer, wherein the tumor does not have any significant alterations (i.e., alterations that result in a change of function) in the indicated gene from the native sequence in the gene and/or transcriptional, translational, and/or splicing control regions of the native gene that result in the cell becoming cancerous, e.g., developing characteristics such as uncontrolled proliferation, immortality, metastatic potential, rapid growth and proliferation rate, decreased cell death/apoptosis, and certain characteristic morphological features. As used herein, a “wild-type” gene is expressed at a level that does not result in the cell becoming cancerous.

Mutations or protein expression levels are preferably detected in a subject sample from the cancer tissue or tumor tissue, e.g., cells, extracellular matrix, and other naturally occurring components associated with the tumor. The mutation or expression level can be detected in a biopsy sample or in a surgical sample after resection of the tumor. The term “sample” as used herein refers to a collection of similar fluids, cells, or tissues isolated from a subject. The term “sample” includes any body fluid (e.g., urine, serum, blood fluids, lymph, gynecological fluids, cystic fluid, ascetic fluid, ocular fluids, and fluids collected by bronchial lavage and/or peritoneal rinsing), ascites, tissue samples (e.g., tumor samples) or a cell from a subject. Other subject samples include tear drops, serum, cerebrospinal fluid, feces, sputum, and cell extracts. In an embodiment, the sample is removed from the subject. In a particular embodiment, the sample is urine or serum. In an embodiment, the sample comprises cells. In another embodiment, the sample does not comprise cells. In certain embodiments, the sample can be the portion of the subject that is imaged. Samples are typically removed from the subject prior to analysis; however, tumor samples can be analyzed in the subject, for example, using imaging or other detection methods.

As used herein, the terms “identify” or “select” refer to a choice in preference to another. In other words, to identify a subject or select a subject is to perform the active step of picking out that particular subject from a group and confirming the identity of the subject by name or other distinguishing feature. It is understood that identifying a subject or selecting a subject as having one or more mutations in one or more genes of interest, having a wild-type gene, or having a change in the expression level of a protein, and can include any of a number of acts including, but not limited to, performing a test and observing a result that is indicative of a subject having a specific mutation; reviewing a test result of a subject and identifying the subject as having a specific mutation; reviewing documentation on a subject stating that the subject has a specific mutation and identifying the subject as the one discussed in the documentation by confirming the identity of the subject e.g., by an identification card, hospital bracelet, asking the subject for his/her name and/or other personal information to confirm the subjects identity.

As already indicated, the methods and procedures for the detections and/or identifications of EGFR, KRAS, BRAF, and/or ALK over-expressions and/or mutations are known in the literature and can be easily carried out by a skilled person. See, e.g., U.S. Pat. No. 7,700,339; U.S. Patent Application Publication No. US2011/0110923; Palmer et al, Biochem. J. (2009), 345-361; Koivunen et al, Clin. Can. Res., 2008, 14, 4275-4283; Anderson, Expert Rev. Mol. Diagn. 11(6), 635-642 (2011); Pinto et al, Cancer Genetics 204 (2011), 439-446; Rekhtman et al; Clin Cancer Res 2012; 18:1167-1176; Massarelli et al, Clin Cancer Res 2007; 13:2890-2896; Lamy et al, Modern Pathology (2011) 24, 1090-1100; Balschun et al, Expert Rev. Mol. Diagn. 11(8), 799-802 (2011); Vakiani et al, J Pathol 2011; 223, 219-229; Okudela et al, Pathology International 2010; 60: 651-660; John et al, Oncogene (2009) 28, S14-S23; and the references cited in the-above identified references. Thresholds of increased expression that constitute an EGFR mutation or an ALK mutation are well known in the art. Moreover, it is generally recognized that once an EGFR mutation is detected in a cancer, the KRAS mutation will be eliminated in the same cancer. Put reversely, if a KRAS mutation is positively identified in a cancer from a subject, it is then unnecessary to engage in any further EGFR related identification. Similar principles can be applied to an ALK mutation in a cancer, that is, if there is an ALK mutation detected in a cancer, it is extremely rare that an EGFR or KRAS mutation will be implicated. Once an ALK mutation is positively identified in a cancer, no further identification is necessary for either an EGFR mutation or for a KRAS mutation in the same cancer.

As used herein, “detecting”, “detection” and the like are understood that an assay performed for identification of a specific analyte in a sample, e.g., a gene or gene product with a mutation, or the expression level of a gene or gene product in a sample, typically as compared to an appropriate control cell or tissue. The specific method of detection used is not a limitation of the invention. The detection method will typically include comparison to an appropriate control sample.

The term “control sample,” as used herein, refers to any clinically relevant comparative sample, including a sample from a healthy subject not afflicted with cancer, a sample from a subject having a less severe or slower progressing cancer than the subject to be assessed, a sample from a subject having some other type of cancer or disease, a sample from a subject prior to treatment, a sample of non-diseased tissue (e.g., non-tumor tissue), a sample from the same origin and close to the tumor site, and the like. A control sample can be a purified sample, protein, and/or nucleic acid provided with a kit. Such control samples can be diluted, for example, in a dilution series to allow for quantitative measurement of analytes in test samples. A control sample may include a sample derived from one or more subjects. A control sample may also be a sample made at an earlier time point from the subject to be assessed. For example, the control sample could be a sample taken from the subject to be assessed before the onset of the cancer, at an earlier stage of disease, or before the administration of treatment or of a portion of treatment. The control sample may also be a sample from an animal model, or from a tissue or cell lines derived from the animal model, of the cancer. The level of signal detected or protein expression in a control sample that consists of a group of measurements may be determined, e.g., based on any appropriate statistical measure, such as measures of central tendency including average, median, or modal values.

As used herein, the term “refractory” cancer or tumor is understood as a malignancy which is either initially unresponsive to chemo- or radiation therapy, or which becomes unresponsive over time. A cancer refractory to on intervention may not be refractory to all interventions. A refractory cancer is typically not amenable to treatment with surgical interventions.

As used herein, “relapse” is understood as the return of a cancer or the signs and symptoms of a cancer after a period of improvement.

The articles “a”, “an” and “the” are used herein to refer to one or to more than one (i.e. to at least one) of the grammatical object of the article unless otherwise clearly indicated by contrast. By way of example, “an element” means one element or more than one element.

The term “including” is used herein to mean, and is used interchangeably with, the phrase “including but not limited to”.

The term “or” is used herein to mean, and is used interchangeably with, the term “and/or,” unless context clearly indicates otherwise.

The term “such as” is used herein to mean, and is used interchangeably, with the phrase “such as but not limited to”.

As used herein, a “proliferative disorder” or a “hyperproliferative disorder,” and other equivalent terms, means a disease or medical condition involving pathological growth of cells. Proliferative disorders include cancer, smooth muscle cell proliferation, systemic sclerosis, cirrhosis of the liver, adult respiratory distress syndrome, idiopathic cardiomyopathy, lupus erythematosus, retinopathy, (e.g., diabetic retinopathy or other retinopathies), cardiac hyperplasia, reproductive system associated disorders such as benign prostatic hyperplasia and ovarian cysts, pulmonary fibrosis, endometriosis, fibromatosis, harmatomas, lymphangiomatosis, sarcoidosis and desmoid tumors. Non-cancerous proliferative disorders also include hyperproliferation of cells in the skin such as psoriasis and its varied clinical forms, Reiter's syndrome, pityriasis rubra pilaris, hyperproliferative variants of disorders of keratinization (e.g., actinic keratosis, senile keratosis), scleroderma, and the like. In one embodiment, the proliferative disorder is a myeloproliferative disorder. In one aspect, the myeloproliferative disorder is polycythemia vera, idiopathic myelofirbrosis, myelodysplastic syndrome, psoriasis or essential thrombocythemia. In one embodiment, the proliferative disorder expresses JAK2V617F mutation of JAK2. In an aspect of this embodiment, the proliferative disorder is polycythemia vera, idiopathic myelofirbrosis, or essential thrombocythemia. In one aspect, the proliferative disorder is polycythemia vera. As used herein, the term “pharmaceutically acceptable salt” refers to a salt prepared from a compound of formulae (I) or (Ia) or at least one compound from Table 1 or 2 having an acidic functional group, such as a carboxylic acid functional group, and a pharmaceutically acceptable inorganic or organic base. Suitable bases include hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. The term “pharmaceutically acceptable salt” also refers to a salt prepared from a compound of formulae (I) or (Ia) or a compound in Tables 1 or 2 having a basic functional group, such as an amine functional group, and a pharmaceutically acceptable inorganic or organic acid. Suitable acids include hydrogen sulfate, citric acid, acetic acid, oxalic acid, hydrochloric acid (HCl), hydrogen bromide (HBr), hydrogen iodide (HI), nitric acid, hydrogen bisulfide, phosphoric acid, isonicotinic acid, oleic acid, tannic acid, pantothenic acid, saccharic acid, lactic acid, salicylic acid, tartaric acid, bitartratic acid, ascorbic acid, succinic acid, maleic acid, besylic acid, fumaric acid, gluconic acid, glucaronic acid, formic acid, benzoic acid, glutamic acid, methanesulfonic acid, ethanesulfonic acid, benzenesulfonic acid, pamoic acid and p-toluenesulfonic acid.

As used herein, the term “pharmaceutically acceptable solvate,” is a solvate formed from the association of one or more pharmaceutically acceptable solvent molecules to one of the compounds of formulae (I) or (Ia) or a compound in Tables 1 or 2. The term “solvate” includes hydrates, e.g., hemihydrate, monohydrate, dihydrate, trihydrate, tetrahydrate, and the like.

A pharmaceutically acceptable carrier may contain inert ingredients which do not unduly inhibit the biological activity of the compound(s) described herein. The pharmaceutically acceptable carriers should be biocompatible, i.e., non-toxic, non-inflammatory, non-immunogenic and devoid of other undesired reactions upon the administration to a subject. Standard pharmaceutical formulation techniques can be employed, such as those described in REMINGTON, J. P., REMINGTON'S PHARMACEUTICAL SCIENCES (Mack Pub. Co., 17th ed., 1985). Suitable pharmaceutical carriers for parenteral administration include sterile water, physiological saline, bacteriostatic saline (saline containing about 0.9% mg/ml benzyl alcohol), phosphate-buffered saline, Hank's solution, Ringer's-lactate, and the like. Methods for encapsulating compositions, such as in a coating of hard gelatin or cyclodextran, are known in the art. See BAKER, ET AL., CONTROLLED RELEASE OF BIOLOGICAL ACTIVE AGENTS, (John Wiley and Sons, 1986).

As used herein, the term “effective amount” refers to an amount of a compound described herein which is sufficient to reduce or ameliorate the severity, duration, progression, or onset of a disease or disorder, delay onset of a disease or disorder, retard or halt the advancement of a disease or disorder, cause the regression of a disease or disorder, prevent or delay the recurrence, development, onset or progression of a symptom associated with a disease or disorder, or enhance or improve the therapeutic effect(s) of another therapy. In one embodiment of the invention, the disease or disorder is a proliferative disorder. The precise amount of compound administered to a subject will depend on the mode of administration, the type and severity of the disease or condition and on the characteristics of the subject, such as general health, age, sex, body weight and tolerance to drugs. For example, for a proliferative disease or disorder, determination of an effective amount will also depend on the degree, severity and type of cell proliferation. The skilled artisan will be able to determine appropriate dosages depending on these and other factors. When co-administered with other therapeutic agents, e.g., when co-administered with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of the invention being used. In cases where no amount is expressly noted, an effective amount should be assumed. Non-limiting examples of an effective amount of a compound described herein are provided herein below. In a specific embodiment, the invention provides a method of treating, managing, or ameliorating a disease or disorder, e.g. a proliferative disorder, or one or more symptoms thereof, the method comprising administering to a subject in need thereof a dose of the Hsp90 inhibitor at least 150 μg/kg, at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds described herein once every day, once every 2 days, once every 3 days, once every 4 days, once every 5 days, once every 6 days, once every 7 days, once every 8 days, once every 10 days, once every two weeks, once every three weeks, or once a month.

The dosage of an individual CHK inhibitor used in combination therapy may be equal to or lower than the dose of an individual therapeutic agent when given independently to treat, manage, or ameliorate a disease or disorder, or one or more symptoms thereof. In one embodiment, the disease or disorder being treated with a combination therapy is a proliferative disorder. In another embodiment, the proliferative disorder is cancer. The recommended dosages of therapeutic agents currently used for the treatment, management, or amelioration of a disease or disorder, or one or more symptoms thereof, can obtained from any reference in the art. See, e.g., GOODMAN & GILMAN'S THE PHARMACOLOGICAL BASIS OF BASIS OF THERAPEUTICS 9^{TH} ED, (Hardman, et al., Eds., NY: Mc-Graw-Hill (1996)); PHYSICIAN'S DESK REFERENCE 57^{TH} ED. (Medical Economics Co., Inc., Montvale, N.J. (2003)).

As used herein, the terms “treat”, “treatment” and “treating” refer to the reduction or amelioration of the progression, severity and/or duration of a disease or disorder, delay of the onset of a disease or disorder, or the amelioration of one or more symptoms (preferably, one or more discernible symptoms) of a disease or disorder, resulting from the administration of one or more therapies (e.g., one or more therapeutic agents such as a compound of the invention). The terms “treat”, “treatment” and “treating” also encompass the reduction of the risk of developing a disease or disorder, and the delay or inhibition of the recurrence of a disease or disorder. In one embodiment, the disease or disorder being treated is a proliferative disorder such as cancer. In specific embodiments, the terms “treat”, “treatment” and “treating” refer to the amelioration of at least one measurable physical parameter of a disease or disorder, such as growth of a tumor, not necessarily discernible by the patient. In other embodiments the terms “treat”, “treatment” and “treating” refer to the inhibition of the progression of a disease or disorder, e.g., a proliferative disorder, either physically by the stabilization of a discernible symptom, physiologically by the stabilization of a physical parameter, or both. In another embodiment, the terms “treat”, “treatment” and “treating” of a proliferative disease or disorder refers to the reduction or stabilization of tumor size or cancerous cell count, and/or delay of tumor formation. In another embodiment, the terms “treat”, “treating” and “treatment” also encompass the administration of a compound described herein as a prophylactic measure to patients with a predisposition (genetic or environmental) to any disease or disorder described herein.

As used herein, the terms “therapeutic agent” and “therapeutic agents” refer to any agent(s) that can be used in the treatment of a disease or disorder, e.g. a proliferative disorder, or one or more symptoms thereof. In certain embodiments, the term “therapeutic agent” refers to a compound described herein. In certain other embodiments, the term “therapeutic agent” does not refer to a compound described herein. Preferably, a therapeutic agent is an agent that is known to be useful for, or has been or is currently being used for the treatment of a disease or disorder, e.g., a proliferative disorder, or one or more symptoms thereof.

As used herein, the term “synergistic” refers to a combination of a compound described herein and another therapeutic agent, which, when taken together, is more effective than the additive effects of the individual therapies. A synergistic effect of a combination of therapies (e.g., a combination of therapeutic agents) permits the use of lower dosages of one or more of the therapeutic agent(s) and/or less frequent administration of the agent(s) to a subject with a disease or disorder, e.g., a proliferative disorder. The ability to utilize lower the dosage of one or more therapeutic agent and/or to administer the therapeutic agent less frequently reduces the toxicity associated with the administration of the agent to a subject without reducing the efficacy of the therapy in the treatment of a disease or disorder. In addition, a synergistic effect can result in improved efficacy of agents in the prevention, management or treatment of a disease or disorder, e.g. a proliferative disorder. Finally, a synergistic effect of a combination of therapies may avoid or reduce adverse or unwanted side effects associated with the use of either therapeutic agent alone.

As used herein, the phrase “side effects” encompasses unwanted and adverse effects of a therapeutic agent. Side effects are always unwanted, but unwanted effects are not necessarily adverse. An adverse effect from a therapeutic agent might be harmful or uncomfortable or risky to a subject. Side effects include fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra-pyramidal symptoms, akathisia, cardiovascular disturbances and sexual dysfunction.

As used herein, the term “in combination” refers to the use of more than one therapeutic agent. The use of the term “in combination” does not restrict the order in which the therapeutic agents are administered to a subject with a disease or disorder, e.g., a proliferative disorder. A first therapeutic agent, such as a compound described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent, such as an anti-cancer agent, to a subject with a disease or disorder, e.g. a proliferative disorder, such as cancer. In one embodiment, the Hsp90 inhibitor and the CHK inhibitor are dosed on independent schedules. In another embodiment, the Hsp90 inhibitor and the CHK inhibitor are dosed on approximately the same schedule. In another embodiment, the Hsp90 inhibitor and the CHK inhibitor are dosed concurrently or sequentially on the same day.

As used herein, the terms “therapies” and “therapy” can refer to any protocol(s), method(s), and/or agent(s) that can be used in the prevention, treatment, management, or amelioration of a disease or disorder, e.g., a proliferative disorder, or one or more symptoms thereof.

A used herein, a “protocol” includes dosing schedules and dosing regimens. The protocols herein are methods of use and include therapeutic protocols.

As used herein, a composition that “substantially” comprises a compound means that the composition contains more than about 80% by weight, more preferably more than about 90% by weight, even more preferably more than about 95% by weight, and most preferably more than about 97% by weight of the compound.

As used herein, a “racemic mixture” means about 50% of one enantiomer and about 50% of is corresponding enantiomer of the molecule. The combination encompasses all enantiomerically-pure, enantiomerically-enriched, diastereomerically pure, diastereomerically enriched, and racemic mixtures of the compounds described herein. Enantiomeric and diastereomeric mixtures can be resolved into their component enantiomers or diastereomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

The compounds described herein are defined by their chemical structures and/or chemical names. Where a compound is referred to by both a chemical structure and a chemical name, and the chemical structure and the chemical name conflict, the chemical structure is determinative of the compound's identity.

When administered to a subject (e.g., a non-human animal for veterinary use or for improvement of livestock or to a human for clinical use), the compounds described herein are administered in an isolated form, or as the isolated form in a pharmaceutical composition. As used herein, “isolated” means that the compounds described herein are separated from other components of either: (a) a natural source, such as a plant or cell, preferably bacterial culture, or (b) a synthetic organic chemical reaction mixture. Preferably, the compounds described herein are purified via conventional techniques. As used herein, “purified” means that when isolated, the isolate contains at least 95%, preferably at least 98%, of a compound described herein by weight of the isolate either as a mixture of stereoisomers, or as a diastereomeric or enantiomeric pure isolate.

Only those choices and combinations of substituents that result in a stable structure are contemplated. Such choices and combinations will be apparent to those of ordinary skill in the art and may be determined without undue experimentation.

The invention can be understood more fully by reference to the following detailed description and illustrative examples, which are intended to exemplify non-limiting embodiments of the invention.

The methods described herein utilize one or more triazolone compounds listed in Tables 1 or 2, or a compound represented by Formulae (I) or (Ia):

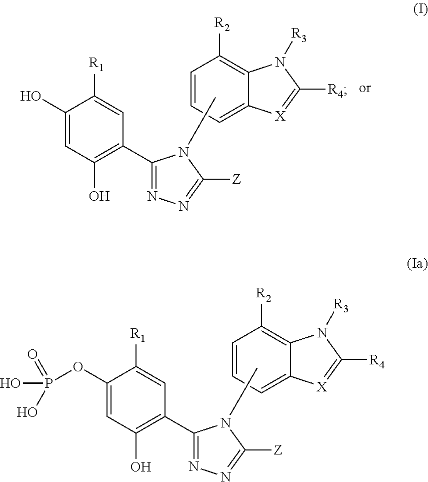

or a tautomer, or a pharmaceutically acceptable salt thereof, wherein:
Z is OH, SH, or NH₂;
X is CR₄ or N;
R₁ is —H, —OH, —SH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanidino, a haloalkyl, a heteroalkyl, an alkoxy or cycloalkoxy, a haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂;
R₂ is —H, —OH, —SH, —NR₇H, —OR₁₅, —SR₁₅, —NHR₁₅, —O(CH₂)ₘOH, —O(CH₂)ₘSH, —O(CH₂)ₘNR₇H, —S(CH₂)ₘOH, —S(CH₂)ₘSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, or —NR₇C(NR₈)NR₁₀R₁₁;
R₃ is —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, a haloalkyl, a heteroalkyl, —C(O)R₇, —(CH₂)ₘC(O)OR₇, —C(O)OR₇, —OC(O)R₇, —C(O)NR₁₀R₁₁, —S(O)ₚR₇, —S(O)ₚOR₇, or —S(O)ₚNR₁₀R₁₁;
R₄ is —H, —OH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, halo, cyano, nitro, guanidino, a haloalkyl, a heteroalkyl, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, —S(O)ₚNR₁₀R₁₁, or R₃ and R₄ taken together with the carbon atoms to which they are attached form an optionally substituted cycloalkenyl, an optionally substituted aryl, an optionally substituted heterocyclyl, or an optionally substituted heteroaryl;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;
R₁₅, for each occurrence, is independently, a lower alkyl;
p, for each occurrence, is, independently, 1 or 2; and
m, for each occurrence, is independently, 1, 2, 3, or 4.

In one embodiment, in formula (I) or (Ia), X is CR₄. In another embodiment, in formula (I) or (Ia), X is N.

In another embodiment, in formula (I) or (Ia), R₁ may be —H, lower alkyl, lower alkoxy, lower cycloalkyl, or lower cycloalkoxy.

In another embodiment, in formula (I) or (Ia), R₁ may be —H, methyl, ethyl, propyl, isopropyl, cyclopropyl, methoxy, ethoxy, propoxy, or cyclopropoxy.

In another embodiment, in formula (I) or (Ia), R₃ may be —H, a lower alkyl, a lower cycloalkyl, —C(O)N(R₂₇)₂, or —C(O)OH, wherein R₂₇ is —H or a lower alkyl.

In another embodiment, in formula (I) or (Ia), R₃ may be —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, or —C(O)N(CH₃)₂.

In one embodiment, R₄ is H or a lower alkyl.

In another embodiment, in formula (I) or (Ia), R₄ may be —H, methyl, ethyl, propyl, isopropyl or cyclopropyl.

In another embodiment, in formula (I) or (Ia), R₁ may be —H, —OH, —SH, —NH₂, a lower alkoxy or a lower alkyl amino.

In another embodiment, in formula (I) or (Ia), R₁ may be —H, —OH, methoxy or ethoxy.

In another embodiment, in formula (I) or (Ia), Z is —OH.

In another embodiment, in formula (I) or (Ia), Z is —SH.

In another embodiment, in formula (I) or (Ia), R₂ may be —H, —OH, —SH, —NH₂, a lower alkoxy or a lower alkyl amino.

In another embodiment, in formula (I) or (Ia), R₂ may be —H, —OH, methoxy, or ethoxy.

In another embodiment, in formula (I) or (Ia), R₁ may be —H, methyl, ethyl, propyl, isopropyl, cyclopropyl, methoxy, ethoxy, propoxy, or cyclopropoxy; R₃ may be of —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, or —C(O)N(CH₃)₂; R₄ may be —H, methyl, ethyl, propyl, isopropyl or cyclopropyl; R₂ may be —H, —OH, —SH, —NH₂, a lower alkoxy or a lower alkyl amino; and Z is OH.

In another embodiment, in formula (I) or (Ia), R₁ is may be —H, methyl, ethyl, propyl, isopropyl, cyclopropyl, methoxy, ethoxy, propoxy, or cyclopropoxy; R₃ is may be —H, methyl, ethyl, n-propyl, isopropyl, cyclopropyl, n-butyl, sec-butyl, tert-butyl, n-pentyl, n-hexyl, —C(O)OH, —(CH₂)ₘC(O)OH, —CH₂OCH₃, —CH₂CH₂OCH₃, or —C(O)N(CH₃)₂; R₄ is may be —H, methyl, ethyl, propyl, isopropyl or cyclopropyl; R₂ is may be —H, —OH, —SH, —NH₂, a lower alkoxy or a lower alkyl amino; and Z is SH.

In another embodiment, the compound is may be:
3-(2,4-dihydroxy-5-ethyl-phenyl)-4-(1,3-dimethyl-indol-5-yl)-5-hydroxy-[1,2,4]triazole,
3-(2,4-dihydroxy-5-isopropyl-phenyl)-4-(1,3-dimethyl-indol-5-yl)-5-hydroxy-[1,2,4]triazole,
3-(2,4-dihydroxy-5-isopropyl-phenyl)-4-(1-methyl-indol-5-yl)-5-hydroxy-[1,2,4]triazole,
3-(2,4-dihydroxy-5-isopropyl-phenyl)-4-(1-isopropyl-indol-4-yl)-5-hydroxy-[1,2,4]triazole,
3-(2,4-dihydroxy-5-isopropyl-phenyl)-4-(1-methyl-indazol-5-yl)-5-mercapto-[1,2,4]triazole,
3-(2,4-dihydroxy-5-isopropyl-phenyl)-4-(1-methyl-indazol-6-yl)-5-mercapto-[1,2,4]triazole,
3-(2,4-dihydroxyphenyl)-4-(1-ethyl-indol-4-yl)-5-mercapto-[1,2,4]triazole,
3-(2,4-dihydroxyphenyl)-4-(1-isopropyl-indol-4-yl)-5-mercapto-[1,2,4]triazole,
3-(2,4-dihydroxyphenyl)-4-(indol-4-yl)-5-mercapto-[1,2,4]triazole,
3-(2,4-dihydroxyphenyl)-4-(1-methoxyethyl-indol-4-yl)-5-mercapto-[1,2,4]triazole,
3-(2,4-dihydroxy-5-ethyl-phenyl)-4-(1-isopropyl-indol-4-yl)-5-mercapto-[1,2,4]triazole,
3-(2,4-dihydroxyphenyl)-4-(1-dimethylcarbamoyl-indol-4-yl)-5-mercapto-[1,2,4]triazole,
3-(2,4-dihydroxy-5-ethyl-phenyl)-4-(1-propyl-indol-4-yl)-5-mercapto-[1,2,4]triazole,
3-(2,4-dihydroxy-5-ethyl-phenyl)-4-(1,2,3-trimethyl-indol-5-yl)-5-mercapto-[1,2,4]triazole,
3-(2,4-dihydroxy-5-ethyl-phenyl)-4-(2,3-dimethyl-indol-5-yl)-5-mercapto-[1,2,4]triazole,
3-(2,4-dihydroxy-5-ethyl-phenyl)-4-(1-acetyl-2,3-dimethyl-indol-5-yl)-5-mercapto-[1,2,4]triazole,
3-(2,4-dihydroxy-5-ethyl-phenyl)-4-(1-propyl-2,3-dimethyl-indol-5-yl)-5-mercapto-[1,2,4]triazole,
3-(2,4-dihydroxy-5-ethyl-phenyl)-4-(1-n-butyl-indol-4-yl)-5-mercapto-[1,2,4]triazole,
3-(2,4-dihydroxy-5-ethyl-phenyl)-4-(1-n-pentyl-indol-4-yl)-5-mercapto-[1,2,4]triazole,
3-(2,4-dihydroxy-5-ethyl-phenyl)-4-(1-n-hexyl-indol-4-yl)-5-mercapto-[1,2,4]triazole,
3-(2,4-dihydroxy-5-cyclopropyl-phenyl)-4-(1-(1-methylcyclopropyl)-indol-4-yl)-5-mercapto-[1,2,4]-triazole,
3-(2,4-dihydroxy-5-cyclopropyl-phenyl)-4-(1,2,3-trimethyl-indol-5-yl)-5-mercapto-[1,2,4]triazole,
3-(2,4-dihydroxy-5-ethyl-phenyl)-4-(1-methyl-3-ethyl-indol-5-yl)-5-mercapto-[1,2,4]triazole,
3-(2,4-dihydroxy-5-ethyl-phenyl)-4-(1,3-dimethyl-indol-5-yl)-5-mercapto-[1,2,4]triazole,
3-(2,4-dihydroxy-5-ethyl-phenyl)-4-(1-methyl-3-isopropyl-indol-5-yl)-5-mercapto-[1,2,4]triazole,
3-(2,4-dihydroxy-5-ethyl-phenyl)-4-(1,2-dimethyl-indol-5-yl)-5-mercapto-[1,2,4]triazole,
3-(2,4-dihydroxy-5-ethyl-phenyl)-4-(N-methyl-indol-5-yl)-5-mercapto-[1,2,4]triazole,
3-(2,4-dihydroxy-5-isopropyl-phenyl)-4-(1,3-dimethyl-indol-5-yl)-5-mercapto-[1,2,4]triazole,
3-(2,4-dihydroxy-5-cyclopropyl-phenyl)-4-(1,3-dimethyl-indol-5-yl)-5-mercapto-[1,2,4]triazole,
3-(2,4-dihydroxy-5-cyclopropyl-phenyl)-4-(1-methyl-indol-5-yl)-5-mercapto-[1,2,4]triazole,
3-(2,4-dihydroxy-5-isopropyl-phenyl)-4-(1H-indol-5-yl)-5-mercapto-[1,2,4]triazole,
3-(2,4-dihydroxy-5-ethyl-phenyl)-4-(1,2-dimethyl-indol-5-yl)-5-mercapto-[1,2,4]triazole,
3-(2,4-dihydroxy-5-isopropyl-phenyl)-4-(1-ethyl-indol-5-yl)-5-mercapto-[1,2,4]triazole, or
3-(2,4-dihydroxy-5-isopropyl-phenyl)-4-(1-propyl-indol-5-yl)-5-mercapto-[1,2,4]triazole, or a tautomer or a pharmaceutically acceptable salt thereof.

In another embodiment, the compound is may be
3-(2,4-dihydroxy-5-ethyl-phenyl)-4-(1-ethyl-benzimidazol-4-yl)-5-mercapto-[1,2,4]triazole,
3-(2,4-dihydroxy-5-ethyl-phenyl)-4-(1-ethyl-benzimidazol-4-yl)-5-mercapto-[1,2,4]triazole HCL salt,
3-(2,4-dihydroxy-5-ethyl-phenyl)-4-(2-methyl-3-ethyl-benzimidazol-5-yl)-5-mercapto-[1,2,4]triazole,
3-(2,4-dihydroxy-5-ethyl-phenyl)-4-(1-ethyl-2-methyl-benzimidazol-5-yl)-5-mercapto-[1,2,4]triazole, or
3-(2,4-dihydroxy-5-isopropyl-phenyl)-4-(1-methyl-2-trifluoromethyl-benzimidazol-5-yl)-5-mercapto-[1,2,4]triazole, or a tautomer or a pharmaceutically acceptable salt thereof.

In another embodiment, the compound is may be
5-hydroxy-4-(5-hydroxy-4-(1-methyl-1H-indol-5-yl)-4H-1,2,4-triazol-3-yl)-2-isopropylphenyl dihydrogen phosphate,
sodium 5-hydroxy-4-(5-hydroxy-4-(1-methyl-1H-indol-5-yl)-4H-1,2,4-triazol-3-yl)-2-isopropylphenyl phosphate,
2-(3,4-dimethoxyphenethyl)-5-hydroxy-4-(5-hydroxy-4-(1-methyl-1H-indol-5-yl)-4H-1,2,4-triazol-3-yl)phenyl dihydrogen phosphate,
5-hydroxy-2-isopropyl-4-(5-mercapto-4-(4-methoxybenzyl)-4H-1,2,4-triazol-3-yl)phenyl dihydrogen phosphate,
5-hydroxy-4-(5-hydroxy-4-(4-methoxybenzyl)-4H-1,2,4-triazol-3-yl)-2-isopropylphenyl dihydrogen phosphate, or
4-(4-(1,3-dimethyl-1H-indol-5-yl)-5-hydroxy-4H-1,2,4-triazol-3-yl)-2-ethyl-5-hydroxyphenyl dihydrogen phosphate, or a tautomer or a pharmaceutically acceptable salt thereof.

Hsp90 inhibitory compounds, as well as tautomers or pharmaceutically acceptable salts thereof that may be used in the methods described herein are depicted in Tables 1 or 2.

TABLE 1

| | STRUCTURE | TAUTOMERIC STRUCTURE | NAME |
|---|---|---|---|
| 1 | 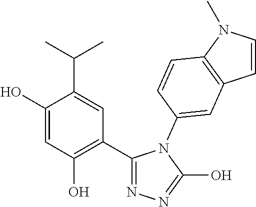 [SMILES: CC(C)c1cc(-c2nnc(O)n2-c2ccc3c(ccn3C)c2)c(O)cc1O] | 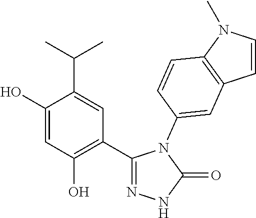 [SMILES: CC(C)c1cc(-c2n[nH]c(=O)n2-c2ccc3c(ccn3C)c2)c(O)cc1O] | 3-(2,4-DIHYDROXY- 5-ISOPROPYL- PHENYL)-4-(1- METHYL-INDOL- 5-YL)-5- HYDROXY- [1,2,4]TRIAZOLE |
| 2 | 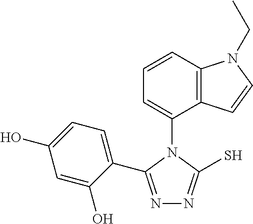 [SMILES: CCn1ccc2c(-n3c(S)nnc3-c3ccc(O)cc3O)cccc21] | 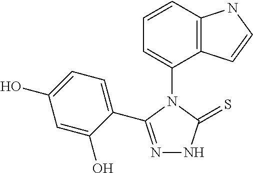 [SMILES: Oc1ccc(-c2n[nH]c(=S)n2-c2cccc3[nH]ccc23)c(O)c1] | 3-(2,4-DIHYDROXY- PHENYL)-4-(1- ETHYL-INDOL- 4-YL)-5- MERCAPTO- [1,2,4]TRIAZOLE |
| 3 | 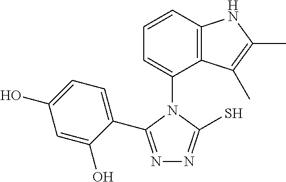 [SMILES: Cc1[nH]c2cccc(-n3c(S)nnc3-c3ccc(O)cc3O)c2c1C] | 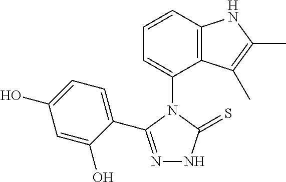 [SMILES: Cc1[nH]c2cccc(-n3c(-c4ccc(O)cc4O)n[nH]c3=S)c2c1C] | 3-(2,4-DIHYDROXY- PHENYL)-4- (2,3-DIMETHYL-1H- INDOL-4- YL)-5-MERCAPTO- [1,2,4]TRIAZOLE |
| 4 | 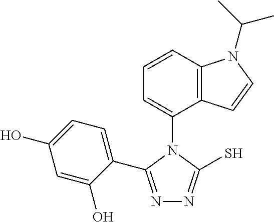 [SMILES: CC(C)n1ccc2c(-n3c(S)nnc3-c3ccc(O)cc3O)cccc21] | 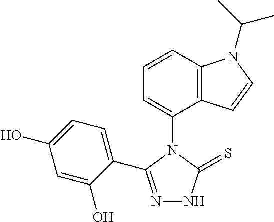 [SMILES: CC(C)n1ccc2c(-n3c(-c4ccc(O)cc4O)n[nH]c3=S)cccc21] | 3-(2,4-DIHYDROXY- PHENYL)-4- (1-ISOPROPYL- INDOL-4-YL)-5- MERCAPTO- [1,2,4]TRIAZOLE |
| 5 | 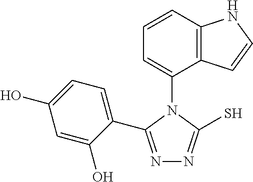 [SMILES: Oc1ccc(-c2nnc(S)n2-c2cccc3[nH]ccc23)c(O)c1] | 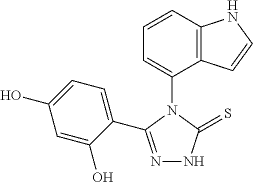 [SMILES: Oc1ccc(-c2n[nH]c(=S)n2-c2cccc3[nH]ccc23)c(O)c1] | 3-(2,4-DIHYDROXY- PHENYL)-4- (INDOL-4-YL)-5- MERCAPTO- [1,2,4]TRIAZOLE |
| 6 | 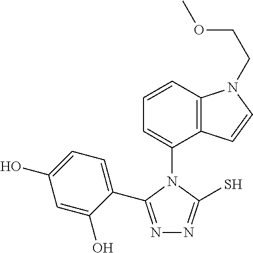 [SMILES: COCCn1ccc2c(-n3c(S)nnc3-c3ccc(O)cc3O)cccc21] | 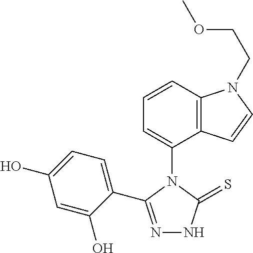 [SMILES: COCCn1ccc2c(-n3c(-c4ccc(O)cc4O)n[nH]c3=S)cccc21] | 3-(2,4-DIHYDROXY- PHENYL)-4- [1-(2-METHOXY- ETHOXY)- INDOL-4-YL]-5- MERCAPTO- [1,2,4]TRIAZOLE |
| 7 | 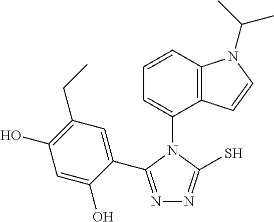 [SMILES: CCc1cc(-c2nnc(S)n2-c2cccc3c2ccn3C(C)C)c(O)cc1O] | 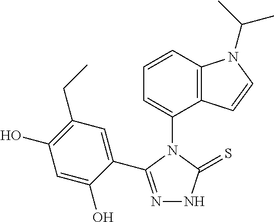 [SMILES: CCc1cc(-c2n[nH]c(=S)n2-c2cccc3c2ccn3C(C)C)c(O)cc1O] | 3-(2,4-DIHYDROXY- 5-ETHYL- PHENYL)-4-(1- ISOPROPYL- INDOL-4-YL)-5- MERCAPTO- [1,2,4]TRIAZOLE |
| 8 | 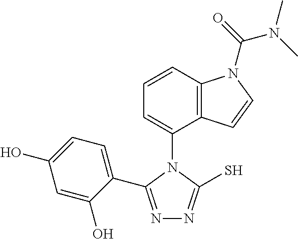 [SMILES: CN(C)C(=O)n1ccc2c(-n3c(S)nnc3-c3ccc(O)cc3O)cccc21] | 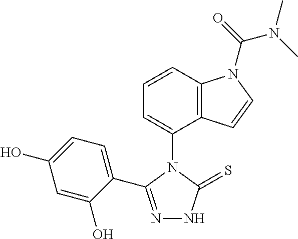 [SMILES: CN(C)C(=O)n1ccc2c(-n3c(-c4ccc(O)cc4O)n[nH]c3=S)cccc21] | 3-(2,4-DIHYDROXY- 5-ETHYL- PHENYL)-4-[1- (DIMETHYL- CARBAMOYL)- INDOL-4-YL]-5- MERCAPTO- [1,2,4]TRIAZOLE |
| 9 | 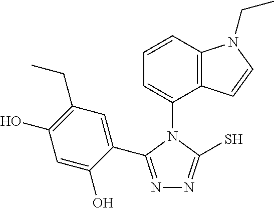 [SMILES: CCc1cc(-c2nnc(S)n2-c2cccc3c2ccn3CC)c(O)cc1O] | 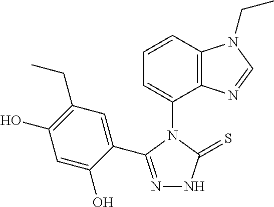 [SMILES: CCc1cc(-c2n[nH]c(=S)n2-c2cccc3c2ncn3CC)c(O)cc1O] | 3-(2,4-DIHYDROXY- 5-ETHYL- PHENYL)-4-(1- ETHYL- BENZOIMIDAZOL- 4-YL)-5- MERCAPTO- [1,2,4]TRIAZOLE |
| 10 | 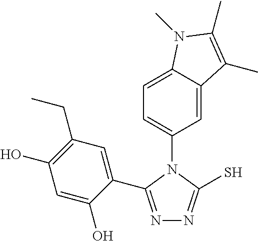 [SMILES: CCc1cc(-c2nnc(S)n2-c2ccc3c(c2)c(C)c(C)n3C)c(O)cc1O] | 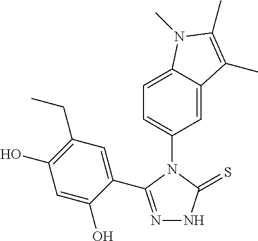 [SMILES: CCc1cc(-c2n[nH]c(=S)n2-c2ccc3c(c2)c(C)c(C)n3C)c(O)cc1O] | 3-(2,4-DIHYDROXY- 5-ETHYL- PHENYL)-4-(1,2,3- TRIMETHYL- INDOL-5-YL)-5- MERCAPTO- [1,2,4]TRIAZOLE |
| 11 | 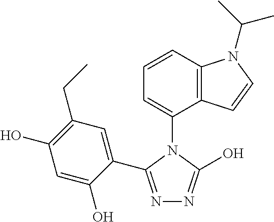 [SMILES: CCc1cc(-c2nnc(O)n2-c2cccc3c2ccn3C(C)C)c(O)cc1O] | 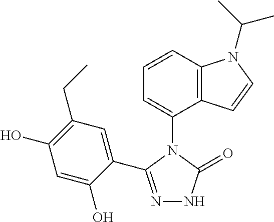 [SMILES: CCc1cc(-c2n[nH]c(=O)n2-c2cccc3c2ccn3C(C)C)c(O)cc1O] | 3-(2,4-DIHYDROXY- 5-ETHYL- PHENYL)-4-(1- ISOPROPYL- INDOL-3-YL)-5- HYDROXY- [1,2,4]TRIAZOLE |
| 12 | 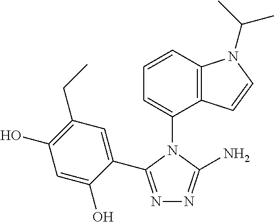 [SMILES: CCc1cc(-c2nnc(N)n2-c2cccc3c2ccn3C(C)C)c(O)cc1O] | 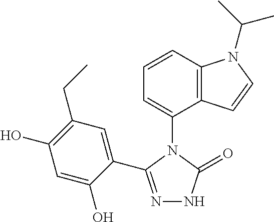 [SMILES: CCc1cc(-c2n[nH]c(=O)n2-c2cccc3c2ccn3C(C)C)c(O)cc1O] | 3-(2,4-DIHYDROXY- 5-ETHYL- PHENYL)-4-(1- ISOPROPYL- INDOL-4-YL)-5- AMINO-[1,2,4] TRIAZOLE |
| 15 | 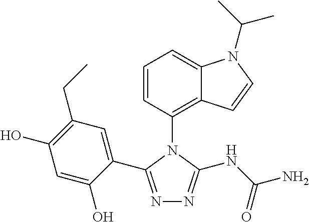 [SMILES: CCc1cc(-c2nnc(NC(N)=O)n2-c2cccc3c2ccn3C(C)C)c(O)cc1O] | | 3-(2,4-DIHYDROXY- 5-ETHYL- PHENYL)-4-(1- ISOPROPYL- INDOL-4-YL)-5- UREIDO-[1,2,4] TRIAZOLE |
| 16 | 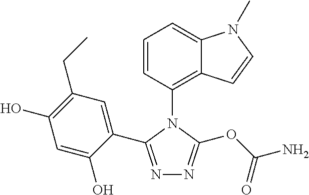 [SMILES: CCc1cc(-c2nnc(OC(N)=O)n2-c2cccc3c2ccn3C)c(O)cc1O] | | 3-(2,4-DIHYDROXY- 5-ETHYL- PHENYL)-4-(1- METHYL-INDOL- 4-YL)-5- CARBAMOYLOXY- [1,2,4]TRIAZOLE |
| 17 | 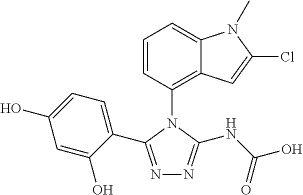 [SMILES: Cn1c(Cl)cc2c(-n3c(NC(=O)O)nnc3-c3ccc(O)cc3O)cccc21] | | 3-(2,4-DIHYDROXY- PHENYL)-4- (1-METHYL-2- CHLORO-INDOL- 4-YL)-5- CARBAMOYLOXY- [1,2,4]TRIAZOLE |
| 18 | 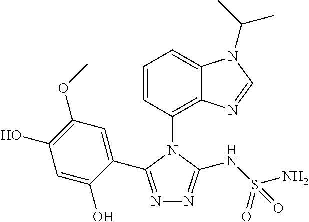 [SMILES: COc1cc(-c2nnc(NS(N)(=O)=O)n2-c2cccc3c2ncn3C(C)C)c(O)cc1O] | | 3-(2,4-DIHYDROXY- 5-METHOXY- PHENYL)-4-(1- ISOPROPYL- BENZOIMIDAZOL- 4-YL)-5- (SULFAMOYL- AMINO)- [1,2,4]TRIAZOLE |
| 20 | 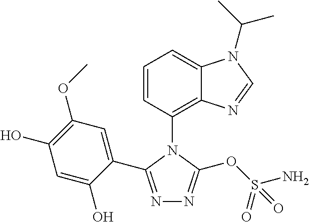 [SMILES: COc1cc(-c2nnc(OS(N)(=O)=O)n2-c2cccc3c2ncn3C(C)C)c(O)cc1O] | | 3-(2,4-DIHYDROXY- 5-METHOXY- PHENYL-4-(1- ISOPROPYL- BENZOIMIDAZOL- 4-YL)-5- (SULFAMOYL- OXY)-[1,2,4] TRIAZOLE |
| 21 | 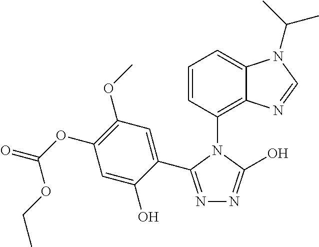 [SMILES: CCOC(=O)Oc1cc(O)c(-c2nnc(O)n2-c2cccc3c2ncn3C(C)C)cc1OC] | 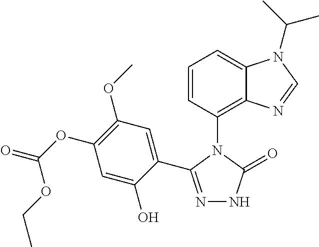 [SMILES: CCOC(=O)Oc1cc(O)c(-c2n[nH]c(=O)n2-c2cccc3c2ncn3C(C)C)cc1OC] | 3-(2-HYDROXY-4- ETHOXY- CARBONYOXY-5- METHOXY- PHENYL-4-(1- ISOPROPYL- BENZOIMIDAZOL- 4-YL)-5- HYDROXY-[1,2,4] TRIAZOLE |
| 22 | 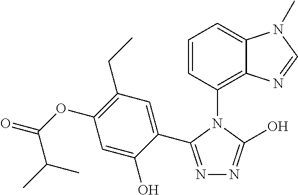 [SMILES: CCc1cc(-c2nnc(O)n2-c2cccc3c2ncn3C)c(O)cc1OC(=O)C(C)C] | 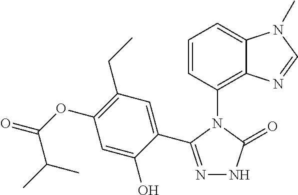 [SMILES: CCc1cc(-c2n[nH]c(=O)n2-c2cccc3c2ncn3C)c(O)cc1OC(=O)C(C)C] | 3-[2-HYDROXY-4- ISOBUTYRYLOXY- 5-ETHYL- PHENYL]-4-(1- METHYL-BENZO- IMIDAZOL-4-YL)- 5-HYDROXY- [1,2,4]TRIAZOLE |
| 23 | 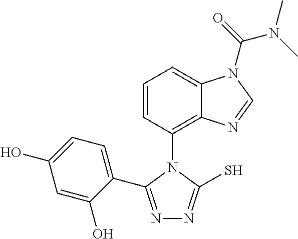 [SMILES: CN(C)C(=O)n1cnc2c(-n3c(S)nnc3-c3ccc(O)cc3O)cccc21] | 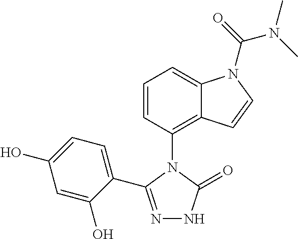 [SMILES: CN(C)C(=O)n1ccc2c(-n3c(-c4ccc(O)cc4O)n[nH]c3=O)cccc21] | 3-(2,4-DIHYDROXY- PHENYL)-4- (1-DIMETHYL- CARBAMOYL- INDOL-4-YL)-5- MERCAPTO- [1,2,4]TRIAZOLE |
| 24 | 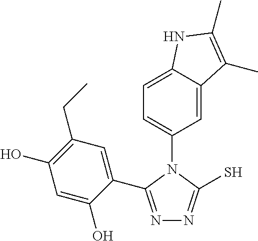 [SMILES: CCc1cc(-c2nnc(S)n2-c2ccc3[nH]c(C)c(C)c3c2)c(O)cc1O] | 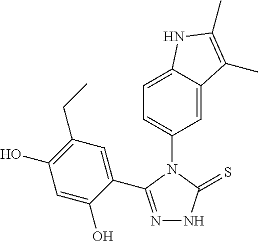 [SMILES: CCc1cc(-c2n[nH]c(=S)n2-c2ccc3[nH]c(C)c(C)c3c2)c(O)cc1O] | 3-(2,4-DIHYDROXY- 5-ETHYL- PHENYL)-4-(2,3- DIMETHYL- INDOL-5-YL)-5- MERCAPTO- [1,2,4]TRIAZOLE |
| 25 | 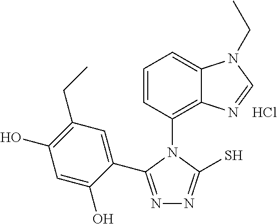 [SMILES: CCc1cc(-c2nnc(S)n2-c2cccc3c2ncn3CC)c(O)cc1O ; Cl] | 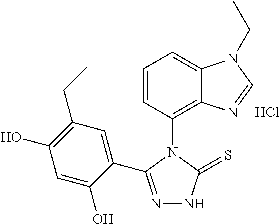 [SMILES: CCc1cc(-c2n[nH]c(=S)n2-c2cccc3c2ncn3CC)c(O)cc1O ; Cl] | 3-(2,4-DIHYDROXY- 5-ETHYL- PHENYL)-4-(1- ETHYL-1H- BENZOIMIDAZOL- 4-YL)-5- MERCAPTO- [1,2,4]TRIAZOLE, |
| 26 | 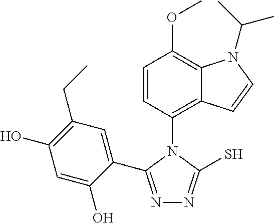 [SMILES: CCc1cc(-c2nnc(S)n2-c2ccc(OC)c3c2ccn3C(C)C)c(O)cc1O] | 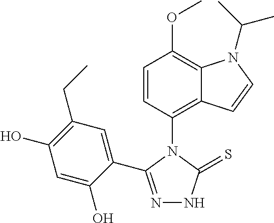 [SMILES: CCc1cc(-c2n[nH]c(=S)n2-c2ccc(OC)c3c2ccn3C(C)C)c(O)cc1O] | 3-(2,4-DIHYDROXY- 5-ETHYL- PHENYL)-4-(1- ISOPROPYL-7- METHOXY- INDOL-4-YL)-5- MERCAPTO- [1,2,4]TRIAZOLE |
| 27 | 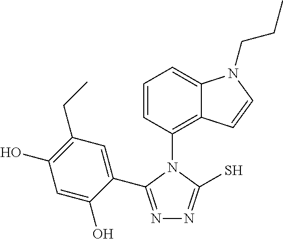 [SMILES: CCCn1ccc2c(-n3c(S)nnc3-c3cc(CC)c(O)cc3O)cccc21] | 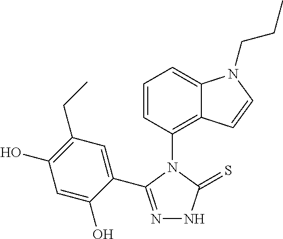 [SMILES: CCCn1ccc2c(-n3c(-c4cc(CC)c(O)cc4O)n[nH]c3=S)cccc21] | 3-(2,4-DIHYDROXY- 5-ETHYL- PHENYL)-4-(1- PROPYL-INDOL- 4-YL)-5- MERCAPTO-[1,2,4] TRIAZOLE |
| 28 | 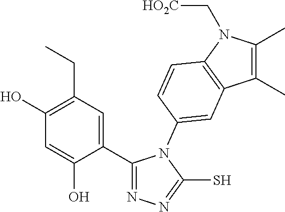 [SMILES: CCc1cc(-c2nnc(S)n2-c2ccc3c(c2)c(C)c(C)n3CC(=O)O)c(O)cc1O] | 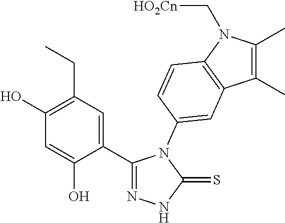 | 3-(2,4-DIHYDROXY- 5-ETHYL- PHENYL-4-(1- ACETYL-2,3- DIMETHYL- INDOL-5-YL)-5- MERCAPTO- [1,2,4]TRIAZOLE |
| 29 | 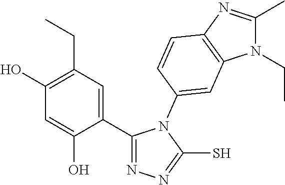 [SMILES: CCc1cc(-c2nnc(S)n2-c2ccc3nc(C)n(CC)c3c2)c(O)cc1O] | 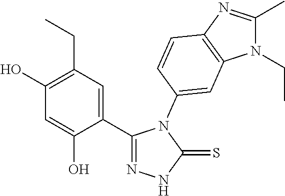 [SMILES: CCc1cc(-c2n[nH]c(=S)n2-c2ccc3nc(C)n(CC)c3c2)c(O)cc1O] | 3-(2,4-DIHYDROXY- 5-ETHYL- PHENYL)-4-(2- METHYL-3- ETHYL- BENZIMIDAZOL- 5-YL)-5- MERCAPTO- [1,2,4]TRIAZOLE |
| 30 | 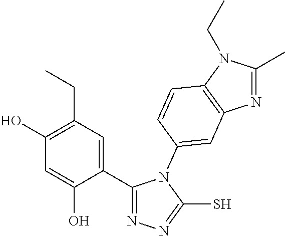 [SMILES: CCc1cc(-c2nnc(S)n2-c2ccc3c(c2)nc(C)n3CC)c(O)cc1O] | 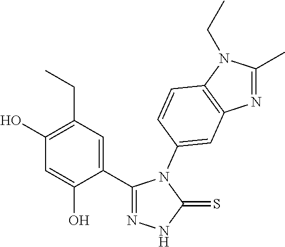 [SMILES: CCc1cc(-c2n[nH]c(=S)n2-c2ccc3c(c2)nc(C)n3CC)c(O)cc1O] | 3-(2,4-DIHYDROXY- 5-ETHYL- PHENYL)-4-(1- ETHYL-2- METHYL- BENZIMIDAZOL- 5-YL)- 5-MERCAPTO- [1,2,4]TRIAZOLE |
| 31 | 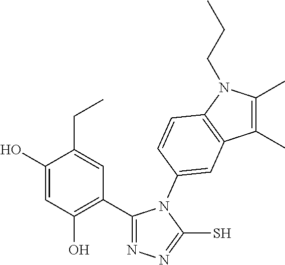 [SMILES: CCCn1c(C)c(C)c2cc(-n3c(S)nnc3-c3cc(CC)c(O)cc3O)ccc21] | 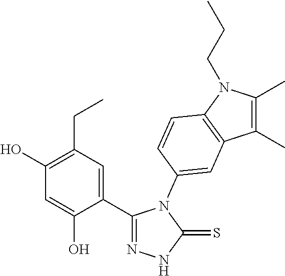 [SMILES: CCCn1c(C)c(C)c2cc(-n3c(-c4cc(CC)c(O)cc4O)n[nH]c3=S)ccc21] | 3-(2,4-DIHYDROXY- 5-ETHYL- PHENYL)-4-(1- PROPYL-2,3- DIMETHYL- INDOL-5-YL)-5- MERCAPTO- [1,2,4]TRIAZOLE |
| 34 | 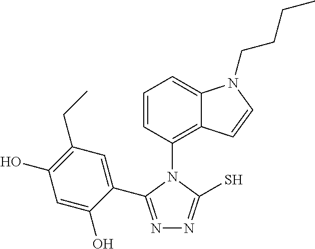 [SMILES: CCCCn1ccc2c(-n3c(S)nnc3-c3cc(CC)c(O)cc3O)cccc21] | 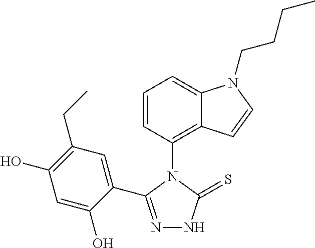 [SMILES: CCCCn1ccc2c(-n3c(-c4cc(CC)c(O)cc4O)n[nH]c3=S)cccc21] | 3-(2,4-DIHYDROXY- 5-ETHYL- PHENYL)-4-(1- N-BUTYL-INDOL- 4-YL)-5- MERCAPTO- [1,2,4]TRIAZOLE |
| 35 | 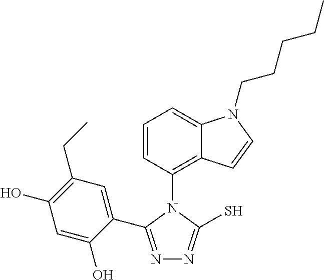 [SMILES: CCCCCn1ccc2c(-n3c(S)nnc3-c3cc(CC)c(O)cc3O)cccc21] | 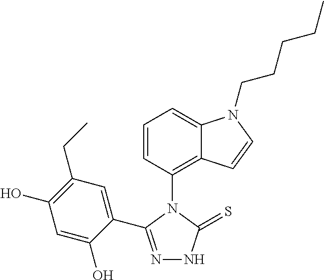 [SMILES: CCCCCn1ccc2c(-n3c(-c4cc(CC)c(O)cc4O)n[nH]c3=S)cccc21] | 3-(2,4-DIHYDROXY- 5-ETHYL- PHENYL)-4- (1-N-PENTYL- INDOL-4-YL)-5- MERCAPTO- [1,2,4]TRIAZOLE |
| 36 | 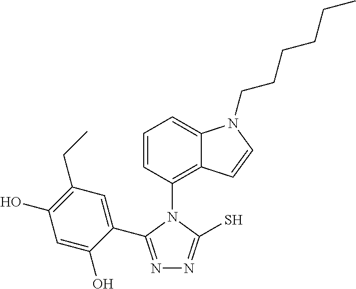 [SMILES: CCCCCCn1ccc2c(-n3c(S)nnc3-c3cc(CC)c(O)cc3O)cccc21] | 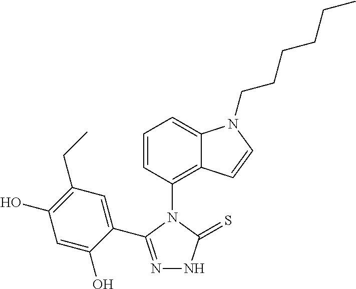 [SMILES: CCCCCCn1ccc2c(-n3c(-c4cc(CC)c(O)cc4O)n[nH]c3=S)cccc21] | 3-(2,4-DIHYDROXY- 5-ETHYL- PHENYL)-4-(1- N-HEXYL-INDOL- 4-YL)-5- MERCAPTO-[1,2,4] TRIAZOLE |
| 37 | 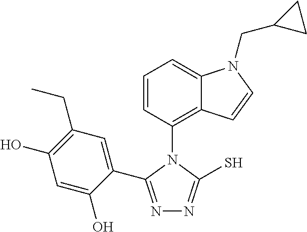 [SMILES: CCc1cc(-c2nnc(S)n2-c2cccc3c2ccn3CC2CC2)c(O)cc1O] | 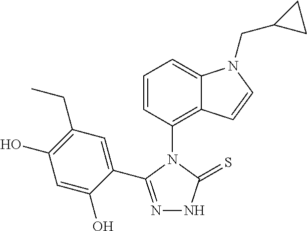 [SMILES: CCc1cc(-c2n[nH]c(=S)n2-c2cccc3c2ccn3CC2CC2)c(O)cc1O] | 3-(2,4-DIHYDROXY- 5-CYCLOPROPYL- PHENYL)-4-(1- (1-METHYL- CYCLOPROPYL)- INDOL-4-YL)-5- MERCAPTO- [1,2,4]TRIAZOLE |
| 38 | 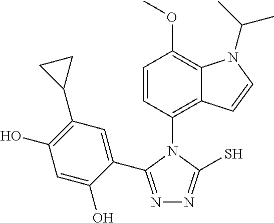 [SMILES: COc1ccc(-n2c(S)nnc2-c2cc(C3CC3)c(O)cc2O)c2ccn(C(C)C)c12] | 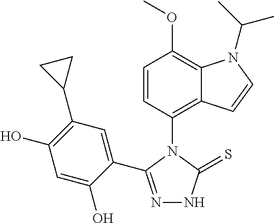 [SMILES: COc1ccc(-n2c(-c3cc(C4CC4)c(O)cc3O)n[nH]c2=S)c2ccn(C(C)C)c12] | 3-(2,4-D1HYDROXY- 5-CYCLOPROPYL- PHENYL)-4-(1- ISOPROPYL-7- METHOXY-INDOL- 4-YL)-5- MERCAPTO- [1,2,4]TRIAZOLE |
| 39 | 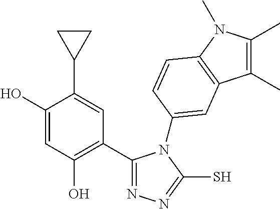 [SMILES: Cc1c(C)n(C)c2ccc(-n3c(S)nnc3-c3cc(C4CC4)c(O)cc3O)cc12] | 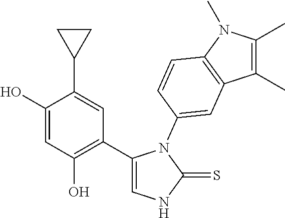 [SMILES: Cc1c(C)n(C)c2ccc(-n3c(-c4cc(C5CC5)c(O)cc4O)c[nH]c3=S)cc12] | 3-(2,4-DIHYDROXY- 5-CYCLOPROPYL- PHENYL)-4- (1,2,3-TRIMETHYL- INDOL-5-YL)- 5-MERCAPTO- [1,2,4]TRIAZOLE |
| 40 | 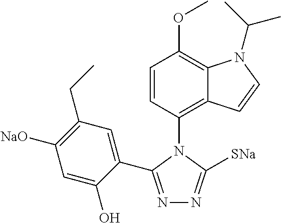 | | 3-(2,4-DIHYDROXY- 5-ETHYL-PHENYL)- 4-(1-ISOPROPYL-7- METHOXY-INDOL- 4-YL)-5- MERCAPTO-[1,2,4] TRIAZOLE DISODIUM SALT |
| 41 | 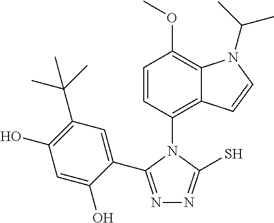 [SMILES: COc1ccc(-n2c(S)nnc2-c2cc(C(C)(C)C)c(O)cc2O)c2ccn(C(C)C)c12] | 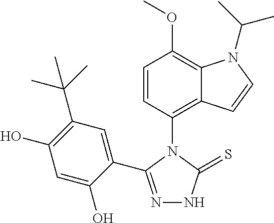 [SMILES: COc1ccc(-n2c(-c3cc(C(C)(C)C)c(O)cc3O)n[nH]c2=S)c2ccn(C(C)C)c12] | 3-(2,4-DIHYDROXY- 5-TERT- BUTYL-PHENYL)-4- (1-ISOPROPYL-7- METHOXY-INDOL- 4-YL)-5- MERCAPTO- [1,2,4]TRIAZOLE |
| 42 | 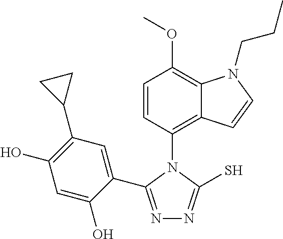 [SMILES: CCCn1ccc2c(-n3c(S)nnc3-c3cc(C4CC4)c(O)cc3O)ccc(OC)c21] | 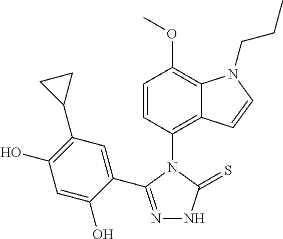 [SMILES: CCCn1ccc2c(-n3c(-c4cc(C5CC5)c(O)cc4O)n[nH]c3=S)ccc(OC)c21] | 3-(2,4-DIHYDROXY- 5-CYCLOPROPYL- PHENYL)-4-(1- PROPYL-7- METHOXY-INDOL- 4-YL)-5- MERCAPTO-[1,2,4] TRIAZOLE |
| 43 | 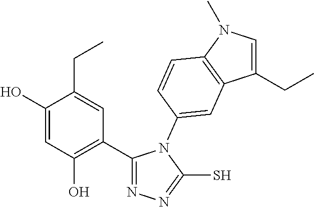 [SMILES: CCc1cc(-c2nnc(S)n2-c2ccc3c(c2)c(CC)cn3C)c(O)cc1O] | 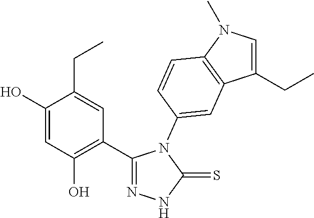 [SMILES: CCc1cc(-c2n[nH]c(=S)n2-c2ccc3c(c2)c(CC)cn3C)c(O)cc1O] | 3-(2,4-DIHYDROXY- 5-ETHYL- PHENYL)-4- (1-METHYL-3- ETHYL-INDOL- 5-YL)-5- MERCAPTO- [1,2,4]TRIAZOLE |
| 44 | 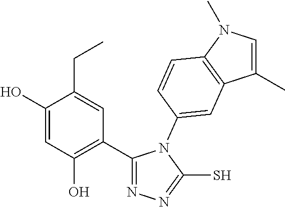 [SMILES: CCc1cc(-c2nnc(S)n2-c2ccc3c(c2)c(C)cn3C)c(O)cc1O] | 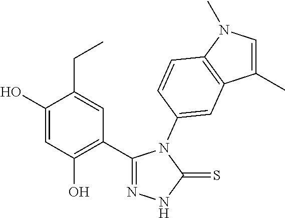 [SMILES: CCc1cc(-c2n[nH]c(=S)n2-c2ccc3c(c2)c(C)cn3C)c(O)cc1O] | 3-(2,4-DIHYDROXY- 5-ETHYL- PHENYL)-4- (1,3-DIMETHYL- INDOL-5-YL)-5- MERCAPTO- [1,2,4]TRIAZOLE |
| 45 | 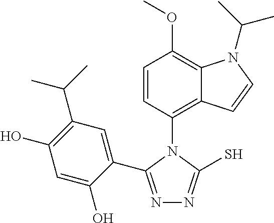 [SMILES: COc1ccc(-n2c(S)nnc2-c2cc(C(C)C)c(O)cc2O)c2ccn(C(C)C)c12] | 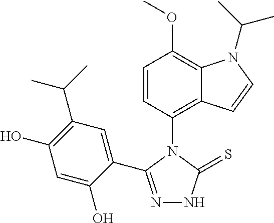 [SMILES: COc1ccc(-n2c(-c3cc(C(C)C)c(O)cc3O)n[nH]c2=S)c2ccn(C(C)C)c12] | 3-(2,4-DIHYDROXY- 5-ISOPROPYL- PHENYL)-4-(1- ISOPROPYL-7- METHOXY-INDOL- 4-YL)-5- MERCAPTO-[1,2,4] TRIAZOLE |
| 46 | 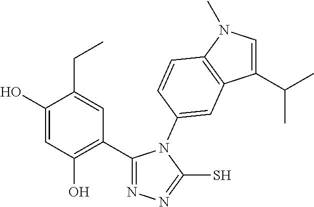 [SMILES: CCc1cc(-c2nnc(S)n2-c2ccc3c(c2)c(C(C)C)cn3C)c(O)cc1O] | 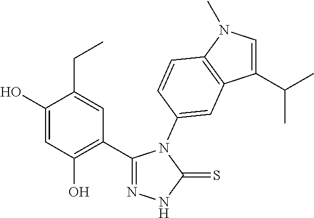 [SMILES: CCc1cc(-c2n[nH]c(=S)n2-c2ccc3c(c2)c(C(C)C)cn3C)c(O)cc1O] | 3-(2,4-DIHYDROXY- 5-ETHYL- PHENYL)-4-(1- METHYL-3- ISOPROPYL- INDOL-5-YL)-5- MERCAPTO- [1,2,4]TRIAZOLE |
| 48 | 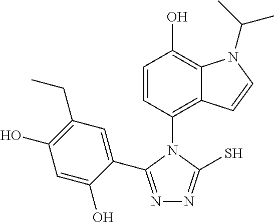 [SMILES: CCc1cc(-c2nnc(S)n2-c2ccc(O)c3c2ccn3C(C)C)c(O)cc1O] | 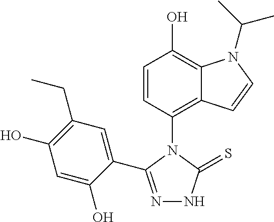 [SMILES: CCc1cc(-c2n[nH]c(=S)n2-c2ccc(O)c3c2ccn3C(C)C)c(O)cc1O] | 3-(2,4-DIHYDROXY- 5-ETHYL- PHENYL)-4-(1- ISOPROPYL-7- HYDROXY- INDOL-4-YL)-5- MERCAPTO- [1,2,4]TRIAZOLE |
| 49 | 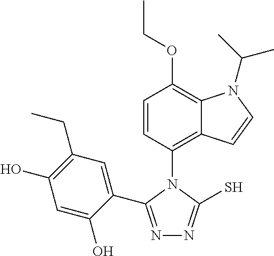 [SMILES: CCOc1ccc(-n2c(S)nnc2-c2cc(CC)c(O)cc2O)c2ccn(C(C)C)c12] | 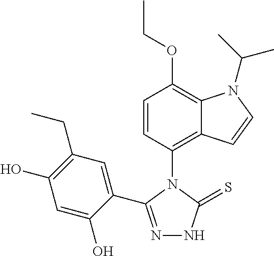 [SMILES: CCOc1ccc(-n2c(-c3cc(CC)c(O)cc3O)n[nH]c2=S)c2ccn(C(C)C)c12] | 3-(2,4-DIHYDROXY- 5-ETHYL- PHENYL)-4-(1- ISOPROPYL-7- ETHOXY-INDOL- 4-YL)-5- MERCAPTO- [1,2,4]TRIAZOLE |
| 50 | 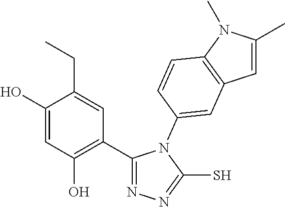 [SMILES: CCc1cc(-c2nnc(S)n2-c2ccc3c(c2)cc(C)n3C)c(O)cc1O] | 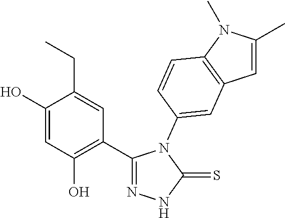 [SMILES: CCc1cc(-c2n[nH]c(=S)n2-c2ccc3c(c2)cc(C)n3C)c(O)cc1O] | 3-(2,4-DIHYDROXY- 5-ETHYL- PHENYL)-4-(1,2- DIMETHYL- INDOL-5-YL)-5- MERCAPTO- [1,2,4]TRIAZOLE |
| 51 | 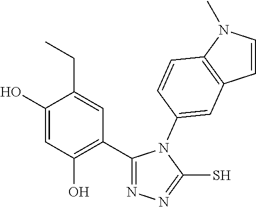 [SMILES: CCc1cc(-c2nnc(S)n2-c2ccc3c(ccn3C)c2)c(O)cc1O] | 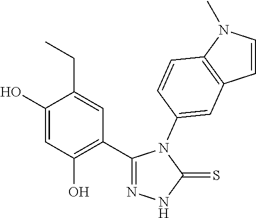 [SMILES: CCc1cc(-c2n[nH]c(=S)n2-c2ccc3c(ccn3C)c2)c(O)cc1O] | 3-(2,4-DIHYDROXY- 5-ETHYL- PHENYL)-4-(N- METHYL-INDOL- 5-YL)-5- MERCAPTO- [1,2,4]TRIAZOLE |
| 55 | 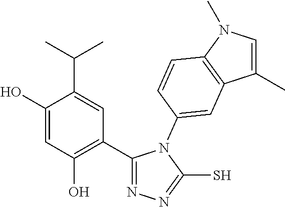 [SMILES: Cc1cn(C)c2ccc(-n3c(S)nnc3-c3cc(C(C)C)c(O)cc3O)cc12] | 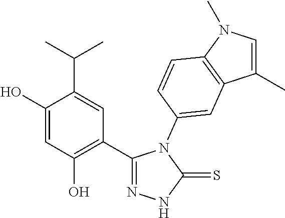 [SMILES: Cc1cn(C)c2ccc(-n3c(-c4cc(C(C)C)c(O)cc4O)n[nH]c3=S)cc12] | 3-(2,4-DIHYDROXY- 5-ISOPROPYL- PHENYL)-4-(1,3- DIMETHYL- INDOL-5-YL)-5- MERCAPTO- [1,2,4]TRIAZOLE |
| 56 | 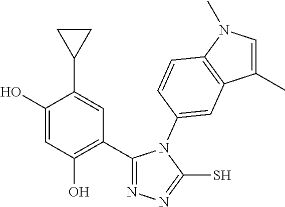 [SMILES: Cc1cn(C)c2ccc(-n3c(S)nnc3-c3cc(C4CC4)c(O)cc3O)cc12] | 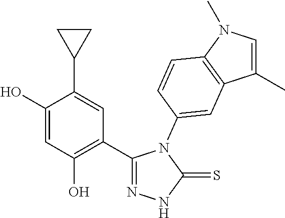 [SMILES: Cc1cn(C)c2ccc(-n3c(-c4cc(C5CC5)c(O)cc4O)n[nH]c3=S)cc12] | 3-(2,4-DIHYDROXY- 5-CYCLOPROPYL- PHENYL)-4-(1,3- DIMETHYL-INDOL- 5-YL)-5- MERCAPTO- [1,2,4]TRIAZOLE |
| 57 | 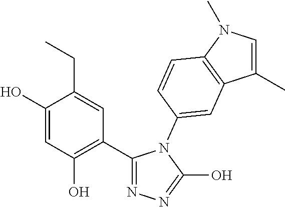 [SMILES: CCc1cc(-c2nnc(O)n2-c2ccc3c(c2)c(C)cn3C)c(O)cc1O] | 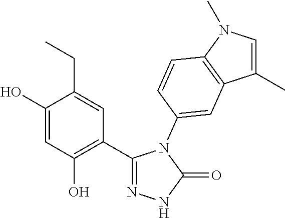 [SMILES: CCc1cc(-c2n[nH]c(=O)n2-c2ccc3c(c2)c(C)cn3C)c(O)cc1O] | 3-(2,4-DIHYDROXY- 5-ETHYL- PHENYL)-4-(1,3- DIMETHYL- INDOL-5-YL)-5- HYDROXY- [1,2,4]TRIAZOLE |
| 58 | 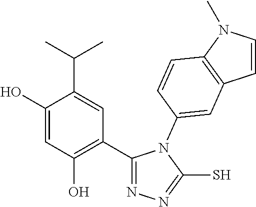 [SMILES: CC(C)c1cc(-c2nnc(S)n2-c2ccc3c(ccn3C)c2)c(O)cc1O] | 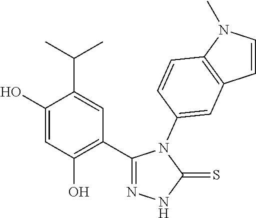 [SMILES: CC(C)c1cc(-c2n[nH]c(=S)n2-c2ccc3c(ccn3C)c2)c(O)cc1O] | 3-(2,4-DIHYDROXY- 5-ISOPROPYL- PHENYL)-4-(N- METHYL-INDOL- 5-YL)-5- MERCAPTO- [1,2,4]TRIAZOLE |
| 59 | 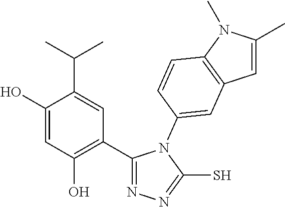 [SMILES: Cc1cc2cc(-n3c(S)nnc3-c3cc(C(C)C)c(O)cc3O)ccc2n1C] | 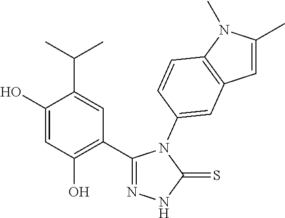 [SMILES: Cc1cc2cc(-n3c(-c4cc(C(C)C)c(O)cc4O)n[nH]c3=S)ccc2n1C] | 3-(2,4-DIHYDROXY-5-ISOPROPYL-PHENYL)-4-(1,2- DIMETHYL- INDOL-5-YL)-5- MERCAPTO- [1,2,4]TRIAZOLE |
| 60 | 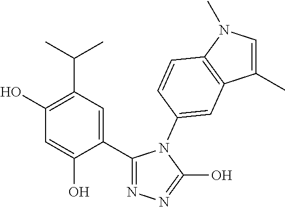 [SMILES: Cc1cn(C)c2ccc(-n3c(O)nnc3-c3cc(C(C)C)c(O)cc3O)cc12] | 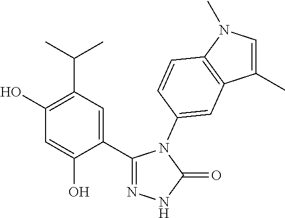 [SMILES: Cc1cn(C)c2ccc(-n3c(-c4cc(C(C)C)c(O)cc4O)n[nH]c3=O)cc12] | 3-(2,4-DIHYDROXY- 5-ISOPROPYL- PHENYL)-4-(1,3- DIMETHYL- INDOL-5-YL)-5- HYDROXY- [1,2,4]TRIAZOLE |
| 62 | 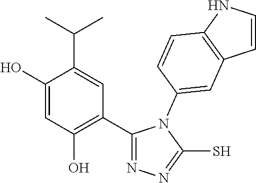 [SMILES: CC(C)c1cc(-c2nnc(S)n2-c2ccc3[nH]ccc3c2)c(O)cc1O] | 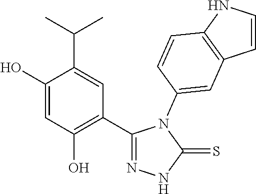 [SMILES: CC(C)c1cc(-c2n[nH]c(=S)n2-c2ccc3[nH]ccc3c2)c(O)cc1O] | 3-(2,4-DIHYDROXY- 5-ISOPROPYL- PHENYL)-4-1H- INDOL-5-YL)-5- MERCAPTO- [1,2,4]TRIAZOLE |
| 63 | 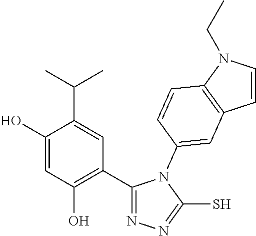 [SMILES: CCn1ccc2cc(-n3c(S)nnc3-c3cc(C(C)C)c(O)cc3O)ccc21] | 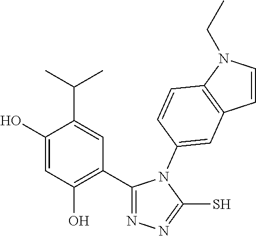 [SMILES: CCn1ccc2cc(-n3c(S)nnc3-c3cc(C(C)C)c(O)cc3O)ccc21] | 3-(2,4-DIHYDROXY- 5-ISOPROPYL- PHENYL)-4-(1- ETHYL-INDOL- 5-YL)-5- MERCAPTO- [1,2,4]TRIAZOLE |
| 64 | 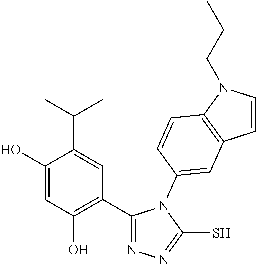 [SMILES: CCCn1ccc2cc(-n3c(S)nnc3-c3cc(C(C)C)c(O)cc3O)ccc21] | 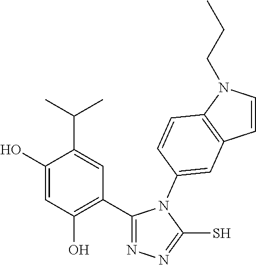 [SMILES: CCCn1ccc2cc(-n3c(S)nnc3-c3cc(C(C)C)c(O)cc3O)ccc21] | 3-(2,4-DIHYDROXY- 5-ISOPROPYL- PHENYL)-4-(1- PROPYL-INDOL- 5-YL)-5- MERCAPTO- [1,2,4]TRIAZOLE |
| 65 | 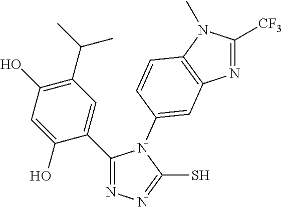 [SMILES: CC(C)c1cc(-c2nnc(S)n2-c2ccc3c(c2)nc(C(F)(F)F)n3C)c(O)cc1O] | 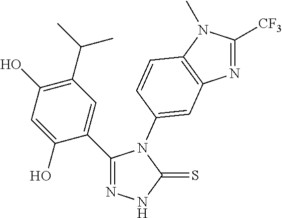 [SMILES: CC(C)c1cc(-c2n[nH]c(=S)n2-c2ccc3c(c2)nc(C(F)(F)F)n3C)c(O)cc1O] | 3-(2,4-DIHYDROXY- 5-ISOPROPYL- PHENYL)-4-(1- METHYL-2- TRIFLUORO- METHYL- BENZIMIDAZOL- 5-YL)-5- MERCAPTO- [1,2,4]TRIAZOLE |
| 66 | 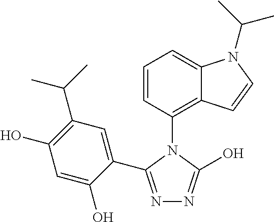 [SMILES: CC(C)c1cc(-c2nnc(O)n2-c2cccc3c2ccn3C(C)C)c(O)cc1O] | 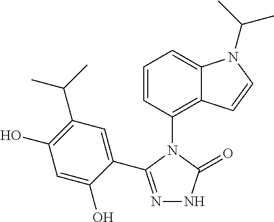 [SMILES: CC(C)c1cc(-c2n[nH]c(=O)n2-c2cccc3c2ccn3C(C)C)c(O)cc1O] | 3-(2,4-DIHYDROXY- 5-ISOPROPYL- PHENYL)-4-(1- ISOPROPYL- INDOL-4-YL)-5- HYDROXY- [1,2,4]TRIAZOLE |

TABLE 2
Compounds according to Formula (Ia)

| No. | Structure | Tautomeric structure | Name |
|---|---|---|---|
| 1a | 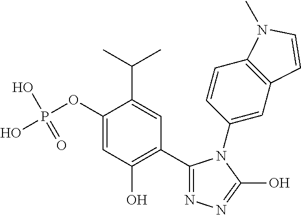 [SMILES: CC(C)c1cc(-c2nnc(O)n2-c2ccc3c(ccn3C)c2)c(O)cc1OP(=O)(O)O] | 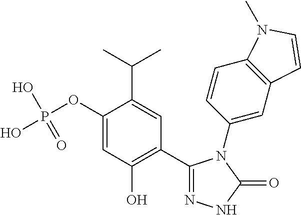 [SMILES: CC(C)c1cc(-c2n[nH]c(=O)n2-c2ccc3c(ccn3C)c2)c(O)cc1OP(=O)(O)O] | 5-hydroxy-4-(5- hydroxy-4-(1-methyl- 1H-indol-5-yl)-4H- 1,2,4-triazol-3-yl)-2- isopropylphenyl dihydrogen phosphate |
| 2a | 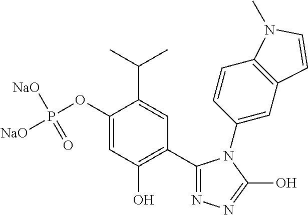 | 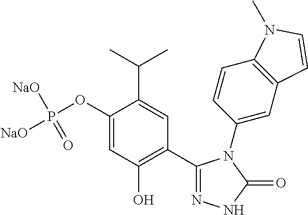 | sodium 5-hydroxy-4- (5-hydroxy-4-(1- methyl-1H-indol-5- yl)-4H-1,2,4-triazol- 3-yl)-2- isopropylphenyl phosphate |
| 3a | 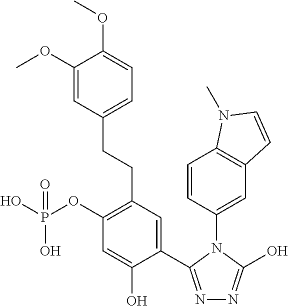 [SMILES: COc1ccc(CCc2cc(-c3nnc(O)n3-c3ccc4c(ccn4C)c3)c(O)cc2OP(=O)(O)O)cc1OC] | 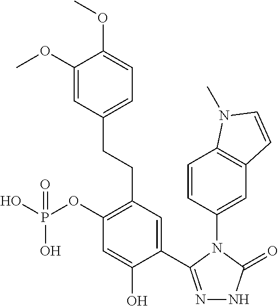 [SMILES: COc1ccc(CCc2cc(-c3n[nH]c(=O)n3-c3ccc4c(ccn4C)c3)c(O)cc2OP(=O)(O)O)cc1OC] | 2-(3,4- dimethoxyphenethyl)- 5-hydroxy-4-(5- hydroxy-4-(1-methyl- 1H-indol-5-yl)-4H- 1,2,4-triazol-3- yl)phenyl dihydrogen phosphate |
| 4a | 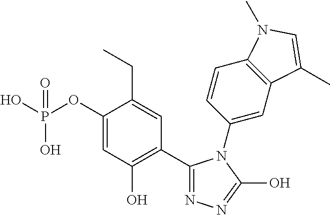 [SMILES: CCc1cc(-c2nnc(O)n2-c2ccc3c(c2)c(C)cn3C)c(O)cc1OP(=O)(O)O] | 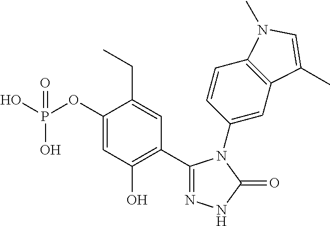 [SMILES: CCc1cc(-c2n[nH]c(=O)n2-c2ccc3c(c2)c(C)cn3C)c(O)cc1OP(=O)(O)O] | 4-(4-(1,3-dimethyl-1H- indol-5-yl)-5- hydroxy-4H-1,2,4- triazol-3-yl)-2-ethyl- 5-hydroxyphenyl dihydrogen phosphate |

The Hsp90 inhibitory compounds used in the disclosed combination methods can be prepared according to the procedures disclosed in U.S. Patent Publication No. 2006/0167070, and WO2009/023211.

These triazolone compounds typically can form a tautomeric structure as shown below and as exemplified by the tautomeric structures shown in Tables 1 and 2:

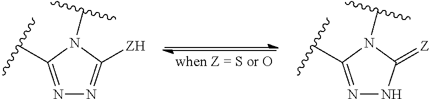

The present invention provides pharmaceutical compositions for the treatment, prophylaxis, and amelioration of proliferative disorders, such as cancer. In a specific embodiment, the combination comprises one or more Hsp90 inhibitors according to formulae (I) or (Ia), or at least one compound from Table 1 or 2, or a tautomer or a pharmaceutically acceptable salt thereof in addition to a CHK inhibitor.

In one embodiment, the pharmaceutical composition includes a single unit dosage form containing both an Hsp90 inhibitor and a CHK inhibitor. Pharmaceutical compositions and dosage forms described herein comprise the two active ingredients in relative amounts and formulated in such a way that a given pharmaceutical composition or dosage form can be used to treat proliferative disorders, such as cancer. Preferred pharmaceutical compositions and dosage forms comprise a compound of formulae (I) or (Ia), or at least one compound from Table 1 or 2, or a tautomer or pharmaceutically acceptable salt thereof, in combination with a CHK inhibitor. In other embodiments, the Hsp90 inhibitor and the CHK inhibitor may be in individual or separate pharmaceutical compositions, depending on the dosing schedules, preferred routes of administration, and available formulations of the two inhibitors. Optionally, these embodiments can also contain one or more additional therapeutic agents.

The pharmaceutical compositions described herein are formulated to be compatible with its intended route of administration. Examples of routes of administration include parenteral, e.g., intravenous, intradermal, subcutaneous, oral, intranasal (e.g., inhalation), transdermal (topical), transmucosal, and rectal administration. In a specific embodiment, the composition is formulated in accordance with routine procedures as a pharmaceutical composition adapted for intravenous, subcutaneous, intramuscular, oral, intranasal or topical administration to human beings. In one embodiment, the composition is formulated in accordance with routine procedures for subcutaneous administration to human beings.

In a specific embodiment, the combination therapies described herein comprise one or more compounds and at least one other therapy which has the same mechanism of action as the compounds. In another specific embodiment, the combination therapies described herein comprise one or more compounds described herein and at least one other therapy which has a different mechanism of action than the compounds. In certain embodiments, the combination therapies described herein improve the therapeutic effect of one or more triazolone compounds described herein by functioning together with the CHK inhibitor to have an additive or synergistic effect. In certain embodiments, the combination therapies described herein reduce the side effects associated with the therapies. In certain embodiments, the combination therapies described herein reduce the effective dosage of one or more of the therapies.

In a specific embodiment, the composition comprising one or more triazolone compounds described herein is administered to a subject, preferably a human, to prevent, treat, manage, or ameliorate cancer, or one or more symptom thereof. In accordance with the invention, the pharmaceutical compositions described herein may also comprise one or more other agents being used, have been used, or are known to be useful in the treatment or amelioration of cancer, particularly colorectal cancer, breast cancer, non-small cell lung cancer, renal cell carcinoma, pancreatic cancer, ovarian cancer, prostate cancer, liver cancer, gliosarcoma, malignant glioma, peritoneal cancer, fallopian tube cancer, rectal cancer, kidney cancer, Hodgkin's lymphoma, bladder cancer, uveal melanoma, gastric cancer, squamous cell carcinoma, cervical cancer, uterine cancer, chronic lymphocytic leukemia, lymphoma, myeloma, Kaposi's sarcoma, urothelial carcinoma, mesothelioma, malignant fibrous histiocytoma, colon cancer, multiple myeloma, gastrointestinal stromal tumor, head and neck cancer, melanoma, or leiomyosarcoma. The pharmaceutical compositions described herein utilize pharmaceutical compositions and dosage forms which comprise one or more excipients. Suitable excipients are well known to those skilled in the art of pharmacy.

The triazolone compounds described herein can be also formulated into or administered by controlled release means or by delivery devices that are well known to those of ordinary skill in the art. Examples include those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566.

The present invention also provides a method of treating a proliferative disorder in a subject, comprising administering to the subject an effective amount of the combination of an Hsp90 inhibitor and a CHK inhibitor as described herein. In one embodiment, the proliferative disorder is cancer. In one aspect of this embodiment, the cancer is colorectal cancer, breast cancer, non-small cell lung cancer, renal cell carcinoma, pancreatic cancer, ovarian cancer, prostate cancer, liver cancer, gliosarcoma, malignant glioma, peritoneal cancer, fallopian tube cancer, rectal cancer, kidney cancer, Hodgkin's lymphoma, bladder cancer, uveal melanoma, gastric cancer, squamous cell carcinoma, cervical cancer, uterine cancer, chronic lymphocytic leukemia, lymphoma, myeloma, Kaposi's sarcoma, urothelial carcinoma, mesothelioma, malignant fibrous histiocytoma, colon cancer, solid tumor, multiple myeloma, gastrointestinal stromal tumor, head and neck cancer, melanoma, or leiomyosarcoma.

Smooth muscle cell proliferation includes hyperproliferation of cells in the vasculature, for example, intimal smooth muscle cell hyperplasia, restenosis and vascular occlusion, particularly stenosis following biologically- or mechanically-mediated vascular injury, e.g., vascular injury associated with angioplasty. Moreover, intimal smooth muscle cell hyperplasia can include hyperplasia in smooth muscle other than the vasculature, e.g., bile duct blockage, bronchial airways of the lung in patients with asthma, in the kidneys of patients with renal interstitial fibrosis, and the like.

In one embodiment, the disclosed method is believed to be effective in treating a subject with non-solid tumors such as multiple myeloma. In another embodiment, the disclosed method is believed to be effective against T-cell leukemia, e.g., as exemplified by Jurkat and CEM cell lines; B-cell leukemia, e.g., as exemplified by the SB cell line; promyelocytes, e.g., as exemplified by the HL-60 cell line; uterine sarcoma, e.g., as exemplified by the MES-SA cell line; monocytic leukemia, e.g., as exemplified by the THP-1 (acute) cell line; and lymphoma, e.g., as exemplified by the U937 cell line.

Other anti-proliferative or anti-cancer therapies may be combined with the compounds described herein to treat proliferative diseases and cancer. Other therapies or anti-cancer agents that may be used in combination with the inventive anti-cancer agents described herein include surgery, radiotherapy (including gamma-radiation, neutron beam radiotherapy, electron beam radiotherapy, proton therapy, brachytherapy, and systemic radioactive isotopes), endocrine therapy, biologic response modifiers (including interferons, interleukins, and tumor necrosis factor (TNF)), hyperthermia and cryotherapy, agents to attenuate any adverse effects (e.g., antiemetics), and other approved chemotherapeutic drugs.

The therapeutic agents of the combination therapies described herein can be administered sequentially or concurrently. In one embodiment, the administration of the Hsp90 inhibitor and the CHK inhibitor are done concurrently. In another embodiment, the administration of the Hsp90 inhibitor and the CHK inhibitor are done separately. In another embodiment, the administration of the Hsp90 inhibitor and the CHK inhibitor are done sequentially. In one embodiment, the administration of the Hsp90 inhibitor and the CHK inhibitor are done until the cancer is cured or stabilized or improved.

In one specific embodiment, the present method includes treating, managing, or ameliorating cancer, or one or more symptoms thereof, comprising administering to a subject in need thereof one or more compounds represented by the structural formulae (I) or (Ia) or a compound in Table 1 or Table 2, in combination with a CHK inhibitor such as 5-(3-fluorophenyl)-3-ureidothiophene-N—[(S)-piperidin-3-yl]-2-carboxamide, 7-nitro-1H-indole-2-carboxylic acid {4-[1-(guanidinohydrazone)-ethyl]-phenyl}-amide, 5-[(8-chloro-3-isoquinolinyl)amino]-3-[(1R)-2-(dimethylamino)-1-methylethoxy]-2-pyrazinecarbonitrile, PF-00477736, CCT241533, or SCH900776, wherein the cancer is colorectal cancer, breast cancer, non-small cell lung cancer, renal cell carcinoma, pancreatic cancer, ovarian cancer, prostate cancer, liver cancer, gliosarcoma, malignant glioma, peritoneal cancer, fallopian tube cancer, rectal cancer, kidney cancer, Hodgkin's lymphoma, bladder cancer, uveal melanoma, gastric cancer, squamous cell carcinoma, cervical cancer, uterine cancer, chronic lymphocytic leukemia, lymphoma, myeloma, Kaposi's sarcoma, urothelial carcinoma, mesothelioma, malignant fibrous histiocytoma, colon cancer, solid tumor, multiple myeloma, gastrointestinal stromal tumor, head and neck cancer, melanoma, or leiomyosarcoma. In one embodiment, the cancer has a KRAS mutation. In one embodiment, the non-small cell lung cancer has a KRAS mutation. In one embodiment, the cancer is ALK positive. In one embodiment, the non-small cell lung cancer is ALK positive. In one embodiment, the cancer has a BRAF mutation. In one embodiment, the melanoma has a BRAF mutation. In another embodiment, the method of treating a subject with cancer includes administering to the subject an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with an effective amount of a CHK inhibitor such as 5-(3-fluorophenyl)-3-ureidothiophene-N—[(S)-piperidin-3-yl]-2-carboxamide, 7-nitro-1H-indole-2-carboxylic acid {4-[1-(guanidinohydrazone)-ethyl]-phenyl}-amide, 5-[(8-chloro-3-isoquinolinyl)amino]-3-[(1R)-2-(dimethylamino)-1-methylethoxy]-2-pyrazinecarbonitrile, PF-00477736, CCT241533, or SCH900776.

In another embodiment, the method of treating a subject with cancer includes administering to the subject an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with an effective amount of AZD7762.

In another embodiment, the method of treating a subject with cancer includes administering to the subject an effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with an effective amount of a CHK inhibitor such as 5-(3-fluorophenyl)-3-ureidothiophene-N—[(S)-piperidin-3-yl]-2-carboxamide, 7-nitro-1H-indole-2-carboxylic acid {4-[1-(guanidinohydrazone)-ethyl]-phenyl}-amide, 5-[(8-chloro-3-isoquinolinyl)amino]-3-[(1R)-2-(dimethylamino)-1-methylethoxy]-2-pyrazinecarbonitrile, PF-00477736, CCT241533, or SCH900776.

In another embodiment, the method of treating a subject with cancer includes administering to the subject an effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with an effective amount of AZD7762.

In another embodiment, the method of treating a subject with cancer includes administering to the subject an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with a CHK inhibitor such as 5-(3-fluorophenyl)-3-ureidothiophene-N—[(S)-piperidin-3-yl]-2-carboxamide, 7-nitro-1H-indole-2-carboxylic acid {4-[1-(guanidinohydrazone)-ethyl]-phenyl}-amide, 5-[(8-chloro-3-isoquinolinyl)amino]-3-[(1R)-2-(dimethylamino)-1-methylethoxy]-2-pyrazinecarbonitrile, PF-00477736, CCT241533, or SCH900776, wherein the cancer is colorectal cancer, breast cancer, non-small cell lung cancer, renal cell carcinoma, pancreatic cancer, ovarian cancer, prostate cancer, liver cancer, gliosarcoma, malignant glioma, peritoneal cancer, fallopian tube cancer, rectal cancer, kidney cancer, Hodgkin's lymphoma, bladder cancer, uveal melanoma, gastric cancer, squamous cell carcinoma, cervical cancer, uterine cancer, chronic lymphocytic leukemia, lymphoma, myeloma, Kaposi's sarcoma, urothelial carcinoma, mesothelioma, malignant fibrous histiocytoma, colon cancer, solid tumor, multiple myeloma, gastrointestinal stromal tumor, head and neck cancer, melanoma, or leiomyosarcoma. In one embodiment, the cancer has a KRAS mutation. In one embodiment, the non-small cell lung cancer has a KRAS mutation. In one embodiment, the cancer is ALK positive. In one embodiment, the non-small cell lung cancer is ALK positive. In one embodiment, the cancer has a BRAF mutation. In one embodiment, the melanoma has a BRAF mutation.

In another embodiment, the method of treating a subject with cancer includes administering to the subject an effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with a CHK inhibitor such as 5-(3-fluorophenyl)-3-ureidothiophene-N—[(S)-piperidin-3-yl]-2-carboxamide, 7-nitro-1H-indole-2-carboxylic acid {4-[1-(guanidinohydrazone)-ethyl]-phenyl}-amide, 5-[(8-chloro-3-isoquinolinyl)amino]-3-[(1R)-2-(dimethylamino)-1-methylethoxy]-2-pyrazinecarbonitrile, PF-00477736, CCT241533, or SCH900776, wherein the cancer is colorectal cancer, breast cancer, non-small cell lung cancer, renal cell carcinoma, pancreatic cancer, ovarian cancer, prostate cancer, liver cancer, gliosarcoma, malignant glioma, peritoneal cancer, fallopian tube cancer, rectal cancer, kidney cancer, Hodgkin's lymphoma, bladder cancer, uveal melanoma, gastric cancer, squamous cell carcinoma, cervical cancer, uterine cancer, chronic lymphocytic leukemia, lymphoma, myeloma, Kaposi's sarcoma, urothelial carcinoma, mesothelioma, malignant fibrous histiocytoma, colon cancer, solid tumor, multiple myeloma, gastrointestinal stromal tumor, head and neck cancer, melanoma, or leiomyosarcoma. In one embodiment, the cancer has a KRAS mutation. In one embodiment, the non-small cell lung cancer has a KRAS mutation. In one embodiment, the cancer is ALK positive. In one embodiment, the non-small cell lung cancer is ALK positive. In one embodiment, the cancer has a BRAF mutation. In one embodiment, the melanoma has a BRAF mutation.

In yet another embodiment, the method of treating a subject with cancer, wherein the subject is being or has been treated with a chemotherapeutic agent, includes administering to the subject an effective amount of a triazolone compound represented by the structural formulae (I) or (Ia) or a compound in Table 1 or Table 2, in combination with a CHK inhibitor such as 5-(3-fluorophenyl)-3-ureidothiophene-N—[(S)-piperidin-3-yl]-2-carboxamide (AZD7762), 7-nitro-1H-indole-2-carboxylic acid {4-[1-(guanidinohydrazone)-ethyl]-phenyl}-amide (PV1019), 5-[(8-chloro-3-isoquinolinyl)amino]-3-[(1R)-2-(dimethylamino)-1-methylethoxy]-2-pyrazinecarbonitrile (SAR-020106), PF-00477736, CCT241533, or SCH900776.

In one embodiment, the method of treating a subject with cancer, wherein the subject is being or has been treated with a chemotherapeutic agent, includes administering to the subject an effective amount of a triazolone compound represented by the structural formulae (I) or (Ia) or a compound in Table 1 or Table 2, in combination with a CHK inhibitor such as 5-(3-fluorophenyl)-3-ureidothiophene-N—[(S)-piperidin-3-yl]-2-carboxamide, 7-nitro-1H-indole-2-carboxylic acid {4-[1-(guanidinohydrazone)-ethyl]-phenyl}-amide, 5-[(8-chloro-3-isoquinolinyl)amino]-3-[(1R)-2-(dimethylamino)-1-methylethoxy]-2-pyrazinecarbonitrile, PF-00477736, CCT241533, or SCH900776, wherein the cancer is colorectal cancer, breast cancer, non-small cell lung cancer, renal cell carcinoma, pancreatic cancer, ovarian cancer, prostate cancer, liver cancer, gliosarcoma, malignant glioma, peritoneal cancer, fallopian tube cancer, rectal cancer, kidney cancer, Hodgkin's lymphoma, bladder cancer, uveal melanoma, gastric cancer, squamous cell carcinoma, cervical cancer, uterine cancer, chronic lymphocytic leukemia, lymphoma, myeloma, Kaposi's sarcoma, urothelial carcinoma, mesothelioma, malignant fibrous histiocytoma, colon cancer, solid tumor, multiple myeloma, gastrointestinal stromal tumor, head and neck cancer, melanoma, or leiomyosarcoma. In one embodiment, the cancer has a KRAS mutation. In one embodiment, the non-small cell lung cancer has a KRAS mutation. In one embodiment, the cancer is ALK positive. In one embodiment, the non-small cell lung cancer is ALK positive. In one embodiment, the cancer has a BRAF mutation. In one embodiment, the melanoma has a BRAF mutation.

In another embodiment, the method of treating a subject with cancer, wherein the subject is being or has been treated with a chemotherapeutic agent, includes administering to the subject an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with a CHK inhibitor such as 5-(3-fluorophenyl)-3-ureidothiophene-N—[(S)-piperidin-3-yl]-2-carboxamide, 7-nitro-1H-indole-2-carboxylic acid {4-[1-(guanidinohydrazone)-ethyl]-phenyl}-amide, 5-[(8-chloro-3-isoquinolinyl)amino]-3-[(1R)-2-(dimethylamino)-1-methylethoxy]-2-pyrazinecarbonitrile, PF-00477736, CCT241533, or SCH900776.

In another embodiment, the method of treating a subject with cancer, wherein the subject is being or has been treated with a chemotherapeutic agent, includes administering to the subject an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with AZD7762.

In another embodiment, the method of treating a subject with cancer, wherein the subject is being or has been treated with a chemotherapeutic agent, includes administering to the subject an effective amount of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with a CHK inhibitor such as 5-(3-fluorophenyl)-3-ureidothiophene-N—[(S)-piperidin-3-yl]-2-carboxamide, 7-nitro-1H-indole-2-carboxylic acid {4-[1-(guanidinohydrazone)-ethyl]-phenyl}-amide, 5-[(8-chloro-3-isoquinolinyl)amino]-3-[(1R)-2-(dimethylamino)-1-methylethoxy]-2-pyrazinecarbonitrile, PF-00477736, CCT241533, or SCH900776.

In another embodiment, the method of treating a subject with cancer, wherein the subject is being or has been treated with a chemotherapeutic agent, includes administering to the subject an effective amount of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with AZD7762.

In one embodiment, the method of treating a subject with cancer, wherein the subject is being or has been treated with a chemotherapeutic agent, includes administering to the subject an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with a CHK inhibitor such as 5-(3-fluorophenyl)-3-ureidothiophene-N—[(S)-piperidin-3-yl]-2-carboxamide, 7-nitro-1H-indole-2-carboxylic acid {4-[1-(guanidinohydrazone)-ethyl]-phenyl}-amide, 5-[(8-chloro-3-isoquinolinyl)amino]-3-[(1R)-2-(dimethylamino)-1-methylethoxy]-2-pyrazinecarbonitrile, PF-00477736, CCT241533, or SCH900776, wherein the cancer is colorectal cancer, breast cancer, non-small cell lung cancer, renal cell carcinoma, pancreatic cancer, ovarian cancer, prostate cancer, liver cancer, gliosarcoma, malignant glioma, peritoneal cancer, fallopian tube cancer, rectal cancer, kidney cancer, Hodgkin's lymphoma, bladder cancer, uveal melanoma, gastric cancer, squamous cell carcinoma, cervical cancer, uterine cancer, chronic lymphocytic leukemia, lymphoma, myeloma, Kaposi's sarcoma, urothelial carcinoma, mesothelioma, malignant fibrous histiocytoma, colon cancer, solid tumor, multiple myeloma, gastrointestinal stromal tumor, head and neck cancer, melanoma, or leiomyosarcoma. In one embodiment, the cancer has a KRAS mutation. In one embodiment, the non-small cell lung cancer has a KRAS mutation. In one embodiment, the cancer is ALK positive. In one embodiment, the non-small cell lung cancer is ALK positive. In one embodiment, the cancer has a BRAF mutation. In one embodiment, the melanoma has a BRAF mutation.

In one embodiment, the method of treating a subject with cancer, wherein the subject is being or has been treated with a chemotherapeutic agent, includes administering to the subject an effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with a CHK inhibitor such as 5-(3-fluorophenyl)-3-ureidothiophene-N—[(S)-piperidin-3-yl]-2-carboxamide, 7-nitro-1H-indole-2-carboxylic acid {4-[1-(guanidinohydrazone)-ethyl]-phenyl}-amide, 5-[(8-chloro-3-isoquinolinyl)amino]-3-[(1R)-2-(dimethylamino)-1-methylethoxy]-2-pyrazinecarbonitrile, PF-00477736, CCT241533, or SCH900776, wherein the cancer is colorectal cancer, breast cancer, non-small cell lung cancer, renal cell carcinoma, pancreatic cancer, ovarian cancer, prostate cancer, liver cancer, gliosarcoma, malignant glioma, peritoneal cancer, fallopian tube cancer, rectal cancer, kidney cancer, Hodgkin's lymphoma, bladder cancer, uveal melanoma, gastric cancer, squamous cell carcinoma, cervical cancer, uterine cancer, chronic lymphocytic leukemia, lymphoma, myeloma, Kaposi's sarcoma, urothelial carcinoma, mesothelioma, malignant fibrous histiocytoma, colon cancer, solid tumor, multiple myeloma, gastrointestinal stromal tumor, head and neck cancer, melanoma, or leiomyosarcoma. In one embodiment, the cancer has a KRAS mutation. In one embodiment, the non-small cell lung cancer has a KRAS mutation. In one embodiment, the cancer is ALK positive. In one embodiment, the non-small cell lung cancer is ALK positive. In one embodiment, the cancer has a BRAF mutation. In one embodiment, the melanoma has a BRAF mutation.

In one embodiment, the method of treating a subject with cancer, wherein the subject has proven refractory to other therapies but is no longer on these therapies, includes administering to the subject an effective amount of a triazolone compound represented by the structural formulae (I) or (Ia) or a compound in Table 1 or Table 2, in combination with a CHK inhibitor such as 5-(3-fluorophenyl)-3-ureidothiophene-N—[(S)-piperidin-3-yl]-2-carboxamide, 7-nitro-1H-indole-2-carboxylic acid {4-[1-(guanidinohydrazone)-ethyl]-phenyl}-amide, 5-[(8-chloro-3-isoquinolinyl)amino]-3-[(1R)-2-(dimethylamino)-1-methylethoxy]-2-pyrazinecarbonitrile, PF-00477736, CCT241533, or SCH900776, wherein the cancer is colorectal cancer, breast cancer, non-small cell lung cancer, renal cell carcinoma, pancreatic cancer, ovarian cancer, prostate cancer, liver cancer, gliosarcoma, malignant glioma, peritoneal cancer, fallopian tube cancer, rectal cancer, kidney cancer, Hodgkin's lymphoma, bladder cancer, uveal melanoma, gastric cancer, squamous cell carcinoma, cervical cancer, uterine cancer, chronic lymphocytic leukemia, lymphoma, myeloma, Kaposi's sarcoma, urothelial carcinoma, mesothelioma, malignant fibrous histiocytoma, colon cancer, solid tumor, multiple myeloma, gastrointestinal stromal tumor, head and neck cancer, melanoma, or leiomyosarcoma. In one embodiment, the cancer has a KRAS mutation. In one embodiment, the non-small cell lung cancer has a KRAS mutation. In one embodiment, the cancer is ALK positive. In one embodiment, the non-small cell lung cancer is ALK positive. In one embodiment, the cancer has a BRAF mutation. In one embodiment, the melanoma has a BRAF mutation.

In another embodiment, the method of treating a subject with cancer, wherein the subject has proven refractory to other therapies but is no longer on these therapies, includes administering to the subject an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with a CHK inhibitor such as 5-(3-fluorophenyl)-3-ureidothiophene-N—[(S)-piperidin-3-yl]-2-carboxamide, 7-nitro-1H-indole-2-carboxylic acid {4-[1-(guanidinohydrazone)-ethyl]-phenyl}-amide, 5-[(8-chloro-3-isoquinolinyl)amino]-3-[(1R)-2-(dimethylamino)-1-methylethoxy]-2-pyrazinecarbonitrile, PF-00477736, CCT241533, or SCH900776.

In another embodiment, the method of treating a subject with cancer, wherein the subject has proven refractory to other therapies but is no longer on these therapies, includes administering to the subject an effective amount of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with AZD7762.

In another embodiment, the method of treating a subject with cancer, wherein the subject has proven refractory to other therapies but is no longer on these therapies, includes administering to the subject an effective amount of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with a CHK inhibitor such as 5-(3-fluorophenyl)-3-ureidothiophene-N—[(S)-piperidin-3-yl]-2-carboxamide, 7-nitro-1H-indole-2-carboxylic acid {4-[1-(guanidinohydrazone)-ethyl]-phenyl}-amide, 5-[(8-chloro-3-isoquinolinyl)amino]-3-[(1R)-2-(dimethylamino)-1-methylethoxy]-2-pyrazinecarbonitrile, PF-00477736, CCT241533, or SCH900776.

In another embodiment, the method of treating a subject with cancer, wherein the subject has proven refractory to other therapies but is no longer on these therapies, includes administering to the subject an effective amount of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with AZD7762.

In one embodiment, the method of treating a subject with cancer, wherein the subject has proven refractory to other therapies but is no longer on these therapies, includes administering to the subject an effective amount of a triazolone compound of 3-(2,4-dihydroxy-5-isopropyl-phenyl)-4-(1-methyl-indol-5-yl)-5-hydroxy-[1,2,4]triazole, or a tautomer, or a pharmaceutically acceptable salt thereof, in combination with a CHK inhibitor such as 5-(3-fluorophenyl)-3-ureidothiophene-N—[(S)-piperidin-3-yl]-2-carboxamide, 7-nitro-1H-indole-2-carboxylic acid {4-[1-(guanidinohydrazone)-ethyl]-phenyl}-amide, 5-[(8-chloro-3-isoquinolinyl)amino]-3-[(1R)-2-(dimethylamino)-1-methylethoxy]-2-pyrazinecarbonitrile, PF-00477736, CCT241533, or SCH900776, wherein the cancer is colorectal cancer, breast cancer, non-small cell lung cancer, renal cell carcinoma, pancreatic cancer, ovarian cancer, prostate cancer, liver cancer, gliosarcoma, malignant glioma, peritoneal cancer, fallopian tube cancer, rectal cancer, kidney cancer, Hodgkin's lymphoma, bladder cancer, uveal melanoma, gastric cancer, squamous cell carcinoma, cervical cancer, uterine cancer, chronic lymphocytic leukemia, lymphoma, myeloma, Kaposi's sarcoma, urothelial carcinoma, mesothelioma, malignant fibrous histiocytoma, colon cancer, solid tumor, multiple myeloma, gastrointestinal stromal tumor, head and neck cancer, melanoma, or leiomyosarcoma. In one embodiment, the cancer has a KRAS mutation. In one embodiment, the non-small cell lung cancer has a KRAS mutation. In one embodiment, the cancer is ALK positive. In one embodiment, the non-small cell lung cancer is ALK positive. In one embodiment, the cancer has a BRAF mutation. In one embodiment, the melanoma has a BRAF mutation.

In one embodiment, the method of treating a subject with cancer, wherein the subject has proven refractory to other therapies but is no longer on these therapies, includes administering to the subject an effective amount of a triazolone compound of 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, in combination with a CHK inhibitor such as 5-(3-fluorophenyl)-3-ureidothiophene-N—[(S)-piperidin-3-yl]-2-carboxamide, 7-nitro-1H-indole-2-carboxylic acid {4-[1-(guanidinohydrazone)-ethyl]-phenyl}-amide, 5-[(8-chloro-3-isoquinolinyl)amino]-3-[(1R)-2-(dimethylamino)-1-methylethoxy]-2-pyrazinecarbonitrile, PF-00477736, CCT241533, or SCH900776, wherein the cancer is colorectal cancer, breast cancer, non-small cell lung cancer, renal cell carcinoma, pancreatic cancer, ovarian cancer, prostate cancer, liver cancer, gliosarcoma, malignant glioma, peritoneal cancer, fallopian tube cancer, rectal cancer, kidney cancer, Hodgkin's lymphoma, bladder cancer, uveal melanoma, gastric cancer, squamous cell carcinoma, cervical cancer, uterine cancer, chronic lymphocytic leukemia, lymphoma, myeloma, Kaposi's sarcoma, urothelial carcinoma, mesothelioma, malignant fibrous histiocytoma, colon cancer, solid tumor, multiple myeloma, gastrointestinal stromal tumor, head and neck cancer, melanoma, or leiomyosarcoma. In one embodiment, the cancer has a KRAS mutation. In one embodiment, the non-small cell lung cancer has a KRAS mutation. In one embodiment, the cancer is ALK positive. In one embodiment, the non-small cell lung cancer is ALK positive. In one embodiment, the cancer has a BRAF mutation. In one embodiment, the melanoma has a BRAF mutation.

In one further embodiment, the method includes inhibiting the growth of a cancer or tumor cell comprising the steps of: (a) contacting the cell with an effective amount of a compound of formulae (I) or (Ia) or a compound in Table (1) or Table (2), or tautomer or a pharmaceutically acceptable salt thereof; and (b) exposing the cell to an effective amount of a CHK inhibitor such as 5-(3-fluorophenyl)-3-ureidothiophene-N—[(S)-piperidin-3-yl]-2-carboxamide, 7-nitro-1H-indole-2-carboxylic acid {4-[1-(guanidinohydrazone)-ethyl]-phenyl}-amide, 5-[(8-chloro-3-isoquinolinyl)amino]-3-[(1R)-2-(dimethylamino)-1-methylethoxy]-2-pyrazinecarbonitrile, PF-00477736, CCT241533, or SCH900776.

In one further embodiment, the method includes inhibiting the growth of a cancer or tumor cell comprising the steps of: (a) contacting the cell with an effective amount of a compound of -(2,4-dihydroxy-5-isopropyl-phenyl)-4-(1-methyl-indol-5-yl)-5-hydroxy-[1,2,4]triazole, or a tautomer, or a pharmaceutically acceptable salt thereof; and (b) exposing the cell to an effective amount of a CHK inhibitor such as 5-(3-fluorophenyl)-3-ureidothiophene-N—[(S)-piperidin-3-yl]-2-carboxamide, 7-nitro-1H-indole-2-carboxylic acid {4-[1-(guanidinohydrazone)-ethyl]-phenyl}-amide, 5-[(8-chloro-3-isoquinolinyl)amino]-3-[(1R)-2-(dimethylamino)-1-methylethoxy]-2-pyrazinecarbonitrile, PF-00477736, CCT241533, or SCH900776.

In one further embodiment, the method includes inhibiting the growth of a cancer or tumor cell comprising the steps of: (a) contacting the cell with an effective amount of a compound of -(2,4-dihydroxy-5-isopropyl-phenyl)-4-(1-methyl-indol-5-yl)-5-hydroxy-[1,2,4]triazole, or a tautomer, or a pharmaceutically acceptable salt thereof; and (b) exposing the cell to an effective amount of AZD7762.

In one further embodiment, the method includes inhibiting the growth of a cancer or tumor cell comprising the steps of: (a) contacting the cell with an effective amount of a compound of 5-hydroxy-4-(5-hydroxy-4-(1-methyl-1H-indol-5-yl)-4H-1,2,4-triazol-3-yl)-2-isopropylphenyl dihydrogen phosphate, or tautomer or a pharmaceutically acceptable salt thereof; and (b) exposing the cell to an effective amount of a CHK inhibitor such as 5-(3-fluorophenyl)-3-ureidothiophene-N—[(S)-piperidin-3-yl]-2-carboxamide, 7-nitro-1H-indole-2-carboxylic acid {4-[1-(guanidinohydrazone)-ethyl]-phenyl}-amide, 5-[(8-chloro-3-isoquinolinyl)amino]-3-[(1R)-2-(dimethylamino)-1-methylethoxy]-2-pyrazinecarbonitrile, PF-00477736, CCT241533, or SCH900776.

In one further embodiment, the method includes inhibiting the growth of a cancer or tumor cell comprising the steps of: (a) contacting the cell with an effective amount of a compound of 5-hydroxy-4-(5-hydroxy-4-(1-methyl-1H-indol-5-yl)-4H-1,2,4-triazol-3-yl)-2-isopropylphenyl dihydrogen phosphate, or tautomer or a pharmaceutically acceptable salt thereof; and (b) exposing the cell to an effective amount of AZD7762.

In an embodiment, the invention also provides a method of treating a subject with a cancer with a KRAS mutation including a) identifying a subject with a cancer with a KRAS mutation and b) administering to the subject a combination of an Hsp90 inhibitor according to formulae (I) or (Ia), or at least one compound from Table 1 or 2, or a tautomer or a pharmaceutically acceptable salt thereof with a CHK inhibitor. In one embodiment, the combination is compound 1 (ganetespib) with the CHK inhibitor AZD7762. In one embodiment, the method further comprises administering one or more additional anticancer drugs. In one embodiment, the one or more drugs are BEZ235, AZD6244, AZD8055, SN-38, gemcitabine, camptothecin, docetaxel, cisplatin, oxaliplatin, crizotinib, paclitaxel, trastuzumab, and pemetrexed. In one embodiment, the cancer is non-small cell lung cancer with a KRAS mutation.

In an embodiment, the invention also provides a method of treating a subject with a cancer with an ALK mutation including a) identifying a subject with a cancer with an ALK mutation and b) administering to the subject a combination of an Hsp90 inhibitor according to formulae (I) or (Ia), or at least one compound from Table 1 or 2, or a tautomer or a pharmaceutically acceptable salt thereof with a CHK inhibitor. In one embodiment, the combination is ganetespib with the CHK inhibitor AZD7762. In one embodiment, the method further comprises administering one or more additional anticancer drugs. In one embodiment, the one or more drugs are may be BEZ235, AZD6244, AZD8055, SN-38, gemcitabine, camptothecin, docetaxel, cisplatin, oxaliplatin, crizotinib, paclitaxel, trastuzumab, and pemetrexed. In one embodiment, the cancer is non-small cell lung cancer with an ALK mutation.

In an embodiment, the invention also provides a method of treating a subject with a cancer with an EGFR mutation including a) identifying a subject with a cancer with an EGFR mutation and b) administering to the subject a combination of an Hsp90 inhibitor according to formulae (I) or (Ia), or at least one compound from Table 1 or 2, or a tautomer or a pharmaceutically acceptable salt thereof with a CHK inhibitor. In one embodiment, the combination is ganetespib with the CHK inhibitor AZD7762. In one embodiment, the method further comprises administering one or more additional anticancer drugs. In one embodiment, the one or more drugs are may be BEZ235, AZD6244, AZD8055, SN-38, gemcitabine, camptothecin, docetaxel, cisplatin, oxaliplatin, crizotinib, paclitaxel, trastuzumab, and pemetrexed. In one embodiment, the cancer is non-small cell lung cancer with an EGFR mutation.

In an embodiment, the invention also provides a method of treating a subject with a cancer with a BRAF mutation including a) identifying a subject with a cancer with a BRAF mutation and b) administering to the subject a combination of an Hsp90 inhibitor according to formulae (I) or (Ia), or at least one compound from Table 1 or 2, or a tautomer or a pharmaceutically acceptable salt thereof with a CHK inhibitor. In one embodiment, the combination is ganetespib with the CHK inhibitor AZD235. In one embodiment, the method further comprises administering one or more additional anticancer drugs. In one embodiment, the one or more drugs are may be BEZ235, AZD6244, AZD8055, SN-38, gemcitabine, camptothecin, docetaxel, cisplatin, oxaliplatin, crizotinib, paclitaxel, trastuzumab, and pemetrexed. In one embodiment, the cancer is non-small cell lung cancer with a BRAF mutation. In one embodiment, the cancer is melanoma with a BRAF mutation.

The invention also provides the use of a combination of an Hsp90 inhibitor according to formulae (I) or (Ia), or at least one compound from Table 1 or 2, or a tautomer or a pharmaceutically acceptable salt thereof with a CHK inhibitor for the manufacture of a medicament for the treatment of a subject with cancer. The invention further provides the use of the combination for the manufacture of a medicament for the treatment of a subject with cancer in combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed. In an embodiment, the combination is compound 1 and AZD7762. In one embodiment, the cancer is non-small cell lung cancer. In one embodiment, the non-small cell lung cancer has a KRAS mutation. In one embodiment, the non-small cell lung cancer has an ALK mutation. In one embodiment, the non-small cell lung cancer has a BRAF mutation. In one embodiment, the cancer is breast cancer.

The invention also provides a combination of an Hsp90 inhibitor according to formulae (I) or (Ia), or at least one compound from Table 1 or 2, or a tautomer or a pharmaceutically acceptable salt thereof with a CHK inhibitor for use in treating a subject with cancer. The invention also provides a combination of an Hsp90 inhibitor according to formulae (I) or (Ia), or at least one compound from Table 1 or 2, or a tautomer or a pharmaceutically acceptable salt thereof with a CHK inhibitor for use in treating a subject with cancer in combination with one or more of BEZ235, AZD6244, AZD8055, SN-38, gemcitabine, camptothecin, docetaxel, cisplatin, oxaliplatin, crizotinib, paclitaxel, trastuzumab, and pemetrexed. In an embodiment, the combination is compound 1 and AZD7762. In one embodiment, the cancer is non-small cell lung cancer. In one embodiment, the non-small cell lung cancer has a KRAS mutation. In one embodiment, the non-small cell lung cancer has an ALK mutation. In one embodiment, the non-small cell lung cancer has a BRAF mutation. In one embodiment, the cancer is breast cancer.

In general, the recommended daily dose range of a triazolone compound for the conditions described herein lie within the range of from about 0.01 mg to about 1000 mg per day, given as a single once-a-day dose preferably as divided doses throughout a day. In one embodiment, the daily dose is administered twice daily in equally divided doses. Specifically, a daily dose range should be from about 5 mg to about 500 mg per day, more specifically, between about 10 mg and about 200 mg per day. In managing the patient, the therapy should be initiated at a lower dose, perhaps about 1 mg to about 25 mg, and increased if necessary up to about 200 mg to about 1000 mg per day as either a single dose or divided doses, depending on the patient's global response. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with individual patient response.

Different therapeutically effective amounts may be applicable for different cancers, as will be readily known by those of ordinary skill in the art. Similarly, amounts sufficient to prevent, manage, treat or ameliorate such cancers, but insufficient to cause, or sufficient to reduce, adverse effects associated with the triazolone compounds described herein are also encompassed by the above described dosage amounts and dose frequency schedules. Further, when a patient is administered multiple dosages of a triazolone compound described herein, not all of the dosages need be the same. For example, the dosage administered to the patient may be increased to improve the prophylactic or therapeutic effect of the compound or it may be decreased to reduce one or more side effects that a particular patient is experiencing.

In a specific embodiment, the dosage of the composition comprising a triazolone compound described herein administered to prevent, treat, manage, or ameliorate cancer, or one or more symptoms thereof in a patient is 150 μg/kg, preferably 250 μg/kg, 500 μg/kg, 1 mg/kg, 5 mg/kg, 10 mg/kg, 25 mg/kg, 50 mg/kg, 75 mg/kg, 100 mg/kg, 125 mg/kg, 150 mg/kg, or 200 mg/kg or more of a patient's body weight. In another embodiment, the dosage of the composition comprising a compound described herein administered to prevent, treat, manage, or ameliorate cancer, or one or more symptoms thereof in a patient is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg. The unit dose can be administered 1, 2, 3, 4 or more times daily, or once every 2, 3, 4, 5, 6 or 7 days, or once weekly, once every two weeks, once every three weeks or once monthly.

In certain embodiments, when the triazolone compounds described herein are administered in combination with a CHK inhibitor, the therapies are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In one embodiment, two or more therapies are administered within the same patient visit.

In certain embodiments, one or more compounds described herein and one or more other the therapies (e.g., therapeutic agents) are cyclically administered. Cycling therapy involves the administration of a first therapy (e.g., a first prophylactic or therapeutic agents) for a period of time, followed by the administration of a second therapy (e.g., a second prophylactic or therapeutic agents) for a period of time, followed by the administration of a third therapy (e.g., a third prophylactic or therapeutic agents) for a period of time and so forth, and repeating this sequential administration, i.e., the cycle in order to reduce the development of resistance to one of the agents, to avoid or reduce the side effects of one of the agents, and/or to improve the efficacy of the treatment.

In certain embodiments, administration of the same compound described herein may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, or 6 months.

In a specific embodiment, a method of preventing, treating, managing, or ameliorating a proliferative disorders, such as cancer, or one or more symptoms thereof, the methods comprising administering to a subject in need thereof a dose of at least 150 μg/kg, preferably at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds described herein once every day, preferably, once every 2 days, once every 3 days, once every 4 days, once every 5 days, once every 6 days, once every 7 days, once every 8 days, once every 10 days, once every two weeks, once every three weeks, or once a month. Alternatively, the dose can be divided into portions (typically equal portions) administered two, three, four or more times a day.

### EXAMPLES

### Materials and Methods

The LNCaP, 22Rv1, DU145 and PC3 human prostate cancer cell lines were all purchased from the American Type Culture Collection (Manassas, Va., USA). Cells were maintained and cultured according to standard techniques at 37° C. in 5% (v/v) CO₂ using culture medium recommended by the supplier. All primary antibodies were purchased from Cell Signaling Technology (Beverly, Mass., USA) with the exception of RAF1 (Santa Cruz Biotechnology, Santa Cruz, Calif., USA), p-EGFR (Tyr1068) (Invitrogen, Carlsbad, Calif., USA) and actin (GE Healthcare, UK). The Hsp90 inhibitors ganetespib and 17-AAG were synthesized at Synta Pharmaceuticals Corp.

### Cell Viability Assays

Cellular viability was assessed using the CellTiter-Glo Luminescent Cell Viability Assay (Promega, Madison, Wis., USA) according to the manufacturer's protocol. Twenty-four hours after plating at 5×10³ cells/well in triplicate in 96-well plates, cells were dosed with graded concentrations of ganetespib or 17-AAG for 72 h. CellTiter-Glo was added (50% v/v) to the cells, and the plates incubated for 10 min prior to luminescent detection in a SpectraMax Plus 384 microplate reader (Molecular Devices, Sunnyvale, Calif., USA). Data were normalized to percent of control and IC₅₀ values used to determine the sensitivity of each line.

### Western Blotting

Prostate cancer cell lines were lysed in RIPA buffer (Cell Signaling Technology, Beverly, Mass. USA). Lysates were clarified by centrifugation and equal amounts of protein resolved by SDS-PAGE before transfer to nitrocellulose membranes. Membranes were blocked with 5% skim milk in TBS with 0.5% Tween and immunoblotted with indicated antibodies. Antigen-antibody complexes were visualized using an Odyssey system (LI-COR, Lincoln, Nebr., USA).

Results of Ganetespib with AZD7762

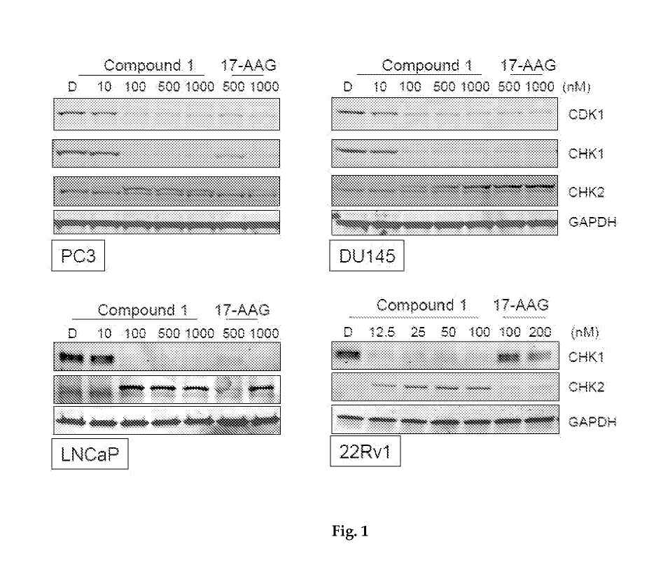

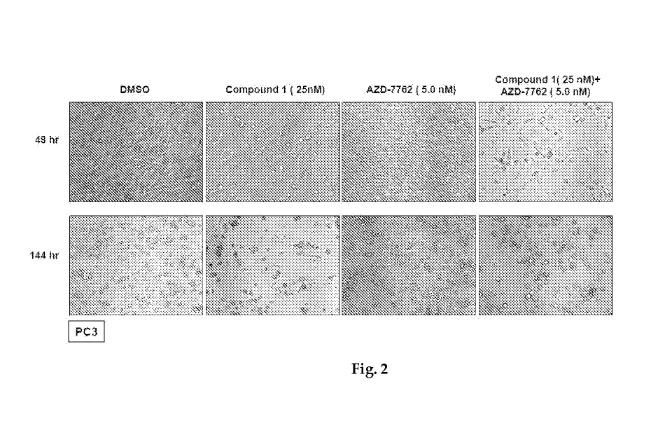

FIGS. 1-2 demonstrate that ganetespib, in combination with CHK inhibitors, shows significant synergy of ganetespib with AZD7762. More particularly, FIG. 1 shows that ganetespib destabilizes the master cell cycle regulator CDK1 and the DNA damage checkpoint CHK1. FIG. 2 shows that inhibition of CHK signaling by AZD-7762 is in synergy with ganetespib in killing PC3 cells. As can be seen, inhibition of Hsp90 activity by ganetespib is highly effective in disrupting CHK activity. Combining ganetespib with AZD7762, a CHK inhibitor, in PC3 cells resulted in a dramatic increase in cell death (FIG. 2).

In summary, ganetespib displayed potent anticancer activity in combination with AZD7762. Without being bound by mechanism, it is suggested that the activity is at least, in part, a result of synergistic effect between ganetespib and the inhibition of CHK pathway.

All publications, patent applications, patents, and other documents cited herein are incorporated by reference in their entirety. In case of conflict, the present specification, including definitions, will control. In addition, the materials, methods, and examples throughout the specification are illustrative only and not intended to be limiting in any way.

What is claimed is:

1. A pharmaceutical composition comprising a CHK inhibitor and an Hsp90 inhibitor according to the following formulae: 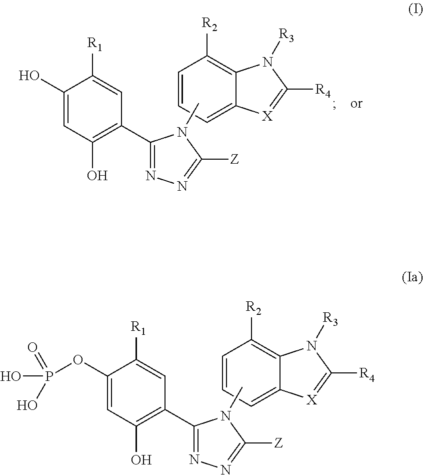
or a tautomer, or a pharmaceutically acceptable salt thereof, wherein:
Z is OH, SH, or NH₂;
X is CR₄ or N;
R₁ is —H, —OH, —SH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, halo, cyano, nitro, guanidino, a haloalkyl, a heteroalkyl, an alkoxy or cycloalkoxy, a haloalkoxy, —NR₁₀R₁₁, —OR₇, —C(O)R₇, —C(O)OR₇, —C(S)R₇, —C(O)SR₇, —C(S)SR₇, —C(S)OR₇, —C(S)NR₁₀R₁₁, —C(NR₈)OR₇, —C(NR₈)R₇, —C(NR₈)NR₁₀R₁₁, —C(NR₈)SR₇, —OC(O)R₇, —OC(O)OR₇, —OC(S)OR₇, —OC(NR₈)OR₇, —SC(O)R₇, —SC(O)OR₇, —SC(NR₈)OR₇, —OC(S)R₇, —SC(S)R₇, —SC(S)OR₇, —OC(O)NR₁₀R₁₁, —OC(S)NR₁₀R₁₁, —OC(NR₈)NR₁₀R₁₁, —SC(O)NR₁₀R₁₁, —SC(NR₈)NR₁₀R₁₁, —SC(S)NR₁₀R₁₁, —OC(NR₈)R₇, —SC(NR₈)R₇, —C(O)NR₁₀R₁₁, —NR₈C(O)R₇, —NR₇C(S)R₇, —NR₇C(S)OR₇, —NR₇C(NR₈)R₇, —NR₇C(O)OR₇, —NR₇C(NR₈)OR₇, —NR₇C(O)NR₁₀R₁₁, —NR₇C(S)NR₁₀R₁₁, —NR₇C(NR₈)NR₁₀R₁₁, —SR₇, —S(O)ₚR₇, —OS(O)ₚR₇, —OS(O)ₚOR₇, —OS(O)ₚNR₁₀R₁₁, —S(O)ₚOR₇, —NR₈S(O)ₚR₇, —NR₇S(O)ₚNR₁₀R₁₁, —NR₇S(O)ₚOR₇, —S(O)ₚNR₁₀R₁₁, —SS(O)ₚR₇, —SS(O)ₚOR₇, —SS(O)ₚNR₁₀R₁₁, —OP(O)(OR₇)₂, or —SP(O)(OR₇)₂;
R₂ is —H, —OH, —SH, —NR₇H, —OR₁₅, —SR₁₅, —NHR₁₅, —O(CH₂)ₘOH, —O(CH₂)ₘSH, —O(CH₂)ₘNR₇H, —S(CH₂)ₘOH, —S(CH₂)ₚSH, —S(CH₂)ₘNR₇H, —OC(O)NR₁₀R₁₁, —SC(O)NR₁₀R₁₁, —NR₇C(O)NR₁₀R₁₁, —OC(O)R₇, —SC(O)R₇, —NR₇C(O)R₇, —OC(O)OR₇, —SC(O)OR₇, —NR₇C(O)OR₇, —OCH₂C(O)R₇, —SCH₂C(O)R₇, —NR₇CH₂C(O)R₇, —OCH₂C(O)OR₇, —SCH₂C(O)OR₇, —NR₇CH₂C(O)OR₇, —OCH₂C(O)NR₁₀R₁₁, —SCH₂C(O)NR₁₀R₁₁, —NR₇CH₂C(O)NR₁₀R₁₁, —OS(O)ₚR₇, —SS(O)ₚR₇, —NR₇S(O)ₚR₇, —OS(O)ₚNR₁₀R₁₁, —SS(O)ₚNR₁₀R₁₁, —NR₇S(O)ₚNR₁₀R₁₁, —OS(O)ₚOR₇, —SS(O)ₚOR₇, —NR₇S(O)ₚOR₇, —OC(S)R₇, —SC(S)R₇, —NR₇C(S)R₇, —OC(S)OR₇, —SC(S)OR₇, —NR₇C(S)OR₇, —OC(S)NR₁₀R₁₁, —SC(S)NR₁₀R₁₁, —NR₇C(S)NR₁₀R₁₁, —OC(NR₈)R₇, —SC(NR₈)R₇, —NR₇C(NR₈)R₇, —OC(NR₈)OR₇, —SC(NR₈)OR₇, —NR₇C(NR₈)OR₇, —OC(NR₈)NR₁₀R₁₁, —SC(NR₈)NR₁₀R₁₁, or —NR₇C(NR₈)NR₁₀R₁₁;
R₃ is —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, a haloalkyl, a heteroalkyl, —C(O)R₇, —(CH₂)ₘC(O)OR₇, —C(O)OR₇, —OC(O)R₇, —C(O)NR₁₀R₁₁, —S(O)ₚR₇, —S(O)ₚOR₇, or —S(O)ₚNR₁₀R₁₁;
R₄ is —H, —OH, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, an optionally substituted heteraralkyl, hydroxyalkyl, alkoxyalkyl, halo, cyano, nitro, guanidino, a haloalkyl, a heteroalkyl, —C(O)R₇, —C(O)OR₇, —OC(O)R₇, —C(O)NR₁₀R₁₁, —NR₈C(O)R₇, —SR₇, —S(O)ₚR₇, —OS(O)ₚR₇, —S(O)ₚOR₇, —NR₈S(O)ₚR₇, —S(O)ₚNR₁₀R₁₁, or R₃ and R₄ taken together with the carbon atoms to which they are attached form an optionally substituted cycloalkenyl, an optionally substituted aryl, an optionally substituted heterocyclyl, or an optionally substituted heteroaryl;
R₇ and R₈, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl;
R₁₀ and R₁₁, for each occurrence, are independently —H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteraralkyl; or R₁₀ and R₁₁, taken together with the nitrogen to which they are attached, form an optionally substituted heterocyclyl or an optionally substituted heteroaryl;
R₁₅, for each occurrence, is independently, a lower alkyl;
p, for each occurrence, is, independently, 1 or 2; and
m, for each occurrence, is independently, 1, 2, 3, or 4.

2. The composition of claim 1, wherein the Hsp90 inhibitor is selected from the group consisting of:
3-(2,4-dihydroxyphenyl)-4-(1-ethyl-indol-4-yl)-5-mercapto-[1,2,4]triazole,
3-(2,4-dihydroxyphenyl)-4-(1-isopropyl-indol-4-yl)-5-mercapto-[1,2,4]triazole,
3-(2,4-dihydroxyphenyl)-4-(indol-4-yl)-5-mercapto-[1,2,4]triazole,
3-(2,4-dihydroxyphenyl)-4-(1-methoxyethyl-indol-4-yl)-5-mercapto-[1,2,4]triazole,
3-(2,4-dihydroxy-5-ethyl-phenyl)-4-(1-isopropyl-indol-4-yl)-5-mercapto-[1,2,4]triazole,
3-(2,4-dihydroxyphenyl)-4-(1-dimethylcarbamoyl-indol-4-yl)-5-mercapto-[1,2,4]triazole,
3-(2,4-dihydroxy-5-ethyl-phenyl)-4-(1-propyl-indol-4-yl)-5-mercapto-[1,2,4]triazole,
3-(2,4-dihydroxy-5-ethyl-phenyl)-4-(1,2,3-trimethyl-indol-5-yl)-5-mercapto-[1,2,4]triazole,
3-(2,4-dihydroxy-5-ethyl-phenyl)-4-(2,3-dimethyl-indol-5-yl)-5-mercapto-[1,2,4]triazole,
3-(2,4-dihydroxy-5-ethyl-phenyl)-4-(1-acetyl-2,3-dimethyl-indol-5-yl)-5-mercapto-[1,2,4]triazole,
3-(2,4-dihydroxy-5-ethyl-phenyl)-4-(1-propyl-2,3-dimethyl-indol-5-yl)-5-mercapto-[1,2,4]triazole,
3-(2,4-dihydroxy-5-ethyl-phenyl)-4-(1-n-butyl-indol-4-yl)-5-mercapto-[1,2,4]triazole,
3-(2,4-dihydroxy-5-ethyl-phenyl)-4-(1-n-pentyl-indol-4-yl)-5-mercapto-[1,2,4]triazole,
3-(2,4-dihydroxy-5-ethyl-phenyl)-4-(1-n-hexyl-indol-4-yl)-5-mercapto-[1,2,4]triazole,
3-(2,4-dihydroxy-5-cyclopropyl-phenyl)-4-(1-(1-methylcyclopropyl)-indol-4-yl)-5-mercapto-[1,2,4]triazole,
3-(2,4-dihydroxy-5-cyclopropyl-phenyl)-4-(1,2,3-trimethyl-indol-5-yl)-5-mercapto-[1,2,4]triazole,
3-(2,4-dihydroxy-5-ethyl-phenyl)-4-(1-methyl-3-ethyl-indol-5-yl)-5-mercapto-[1,2,4]triazole,
3-(2,4-dihydroxy-5-ethyl-phenyl)-4-(1,3-dimethyl-indol-5-yl)-5-mercapto-[1,2,4]triazole,
3-(2,4-dihydroxy-5-ethyl-phenyl)-4-(1-methyl-3-isopropyl-indol-5-yl)-5-mercapto-[1,2,4]triazole,
3-(2,4-dihydroxy-5-ethyl-phenyl)-4-(1,2-dimethyl-indol-5-yl)-5-mercapto-[1,2,4]triazole,
3-(2,4-dihydroxy-5-ethyl-phenyl)-4-(N-methyl-indol-5-yl)-5-mercapto-[1,2,4]triazole,
3-(2,4-dihydroxy-5-isopropyl-phenyl)-4-(1,3-dimethyl-indol-5-yl)-5-mercapto-[1,2,4]triazole,
3-(2,4-dihydroxy-5-cyclopropyl-phenyl)-4-(1,3-dimethyl-indol-5-yl)-5-mercapto-[1,2,4]triazole,
3-(2,4-dihydroxy-5-cyclopropyl-phenyl)-4-(1-methyl-indol-5-yl)-5-mercapto-[1,2,4]triazole,
3-(2,4-dihydroxy-5-isopropyl-phenyl)-4-(1H-indol-5-yl)-5-mercapto-[1,2,4]triazole,
3-(2,4-dihydroxy-5-ethyl-phenyl)-4-(1,2-dimethyl-indol-5-yl)-5-mercapto-[1,2,4]triazole,
3-(2,4-dihydroxy-5-isopropyl-phenyl)-4-(1-ethyl-indol-5-yl)-5-mercapto-[1,2,4]triazole, and
3-(2,4-dihydroxy-5-isopropyl-phenyl)-4-(1-propyl-indol-5-yl)-5-mercapto-[1,2,4]triazole,
5-hydroxy-4-(5-hydroxy-4-(1-methyl-1H-indol-5-yl)-4H-1,2,4-triazol-3-yl)-2-isopropylphenyl dihydrogen phosphate,
sodium 5-hydroxy-4-(5-hydroxy-4-(1-methyl-1H-indol-5-yl)-4H-1,2,4-triazol-3-yl)-2-isopropylphenyl phosphate,
2-(3,4-dimethoxyphenethyl)-5-hydroxy-4-(5-hydroxy-4-(1-methyl-1H-indol-5-yl)-4H-1,2,4-triazol-3-yl)phenyl dihydrogen phosphate,
5-hydroxy-2-isopropyl-4-(5-mercapto-4-(4-methoxybenzyl)-4H-1,2,4-triazol-3-yl)phenyl dihydrogen phosphate,
5-hydroxy-4-(5-hydroxy-4-(4-methoxybenzyl)-4H-1,2,4-triazol-3-yl)-2-isopropylphenyl dihydrogen phosphate,
4-(4-(1,3-dimethyl-1H-indol-5-yl)-5-hydroxy-4H-1,2,4-triazol-3-yl)-2-ethyl-5-hydroxyphenyl dihydrogen phosphate, and
or a tautomer or a pharmaceutically acceptable salt thereof.

3. The composition of claim 1, wherein the Hsp90 inhibitor is selected from the group consisting of:
3-(2,4-Dihydroxy-5-ethyl-phenyl)-4-(1-isopropyl-7-methoxy-indol-4-yl)-5-mercapto-[1,2,4]triazole;
3-(2,4-dihydroxy-5-isopropyl-phenyl)-4-(N-methyl-indol-5-yl)-5-mercapto-[1,2,4]triazole; and
3-(2,4-dihydroxy-5-isopropyl-phenyl)-4-(1-methyl-indol-5-yl)-5-hydroxy-[1,2,4]triazole;
or a tautomer or pharmaceutically acceptable salt thereof.

4. The composition of claim 1, wherein the Hsp90 inhibitor is 3-(2,4-dihydroxy-5-isopropyl-phenyl)-4-(1-methyl-indol-5-yl)-5-hydroxy-[1,2,4]triazole or a tautomer or a pharmaceutically acceptable salt thereof.

5. The composition of claim 1, wherein the Hsp90 inhibitor is 5-hydroxy-4-(5-hydroxy-4-(1-methyl-1H-indol-5-yl)-4H-1,2,4-triazol-3-yl)-2-isopropylphenyl dihydrogen phosphate, or a tautomer or a pharmaceutically acceptable salt thereof.

6. The composition according to claim 1, wherein the CHK inhibitor is selected from the group consisting of 5-(3-fluorophenyl)-3-ureidothiophene-N—[(S)-piperidin-3-yl]-2-carboxamide, 7-nitro-1H-indole-2-carboxylic acid {4-[1-(guanidinohydrazone)-ethyl]-phenyl}-amide, 5-[(8-chloro-3-isoquinolinyl)amino]-3-[(1R)-2-(dimethylamino)-1-methylethoxy]-2-pyrazinecarbonitrile, PF-00477736, CCT241533, and SCH900776.

7. The composition according to claim 6, wherein the CHK inhibitor is 5-(3-fluorophenyl)-3-ureidothiophene-N—[(S)-piperidin-3-yl]-2-carboxamide.

8. The composition according to claim 1, wherein the Hsp90 inhibitor is 3-(2,4-dihydroxy-5-isopropyl-phenyl)-4-(1-methyl-indol-5-yl)-5-hydroxy-[1,2,4]triazole, or a tautomer or a pharmaceutically acceptable salt thereof, and the CHK inhibitor is 5-(3-fluorophenyl)-3-ureidothiophene-N—[(S)-piperidin-3-yl]-2-carboxamide.

9. The composition according to claim 1, wherein the Hsp90 inhibitor is 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, and the CHK inhibitor is 5-(3-fluorophenyl)-3-ureidothiophene-N—[(S)-piperidin-3-yl]-2-carboxamide.

10. A method of treating a proliferative disorder in a subject, comprising administering to a subject an effective amount of the composition of claim 1.

11. The method of claim 10, wherein the proliferative disorder is cancer.

12. The method of claim 11, wherein the cancer is selected from the group consisting of colorectal cancer, breast cancer, non-small cell lung cancer, renal cell carcinoma, pancreatic cancer, ovarian cancer, prostate cancer, liver cancer, gliosarcoma, malignant glioma, peritoneal cancer, fallopian tube cancer, rectal cancer, kidney cancer, Hodgkin's lymphoma, bladder cancer, uveal melanoma, gastric cancer, squamous cell carcinoma, cervical cancer, uterine cancer, chronic lymphocytic leukemia, lymphoma, myeloma, Kaposi's sarcoma, urothelial carcinoma, mesothelioma, malignant fibrous histiocytoma, colon cancer, solid tumor, multiple myeloma, gastrointestinal stromal tumor, head and neck cancer, melanoma, and leiomyosarcoma.

13. The method of claim 12, wherein the cancer is selected from the group consisting of non-small cell lung cancer, colon cancer, solid tumor, multiple myeloma, colorectal cancer, pancreatic cancer, prostate cancer, breast cancer, and melanoma.

14-18. (canceled)

19. The method of claim 13, wherein the cancer has a KRAS, an ALK, a BRAF, or an EGFR mutation.

20. The method of claim 10, wherein the subject is a human.

21. A method of inhibiting the growth of a cancer or tumor cell in a subject, the method comprising the steps of: (a) contacting the cell with an effective amount of a compound of formulae (I) or (Ia) as defined in claim 1, and (b) exposing the cell to an effective amount of a CHK inhibitor, wherein the CHK inhibitor is selected from the group consisting of 5-(3-fluorophenyl)-3-ureidothiophene-N—[(S)-piperidin-3-yl]-2-carboxamide, 7-nitro-1H-indole-2-carboxylic acid {4-[1-(guanidinohydrazone)-ethyl]-phenyl}-amide, 5-[(8-chloro-3-isoquinolinyl)amino]-3-[(1R)-2-(dimethylamino)-1-methylethoxy]-2-pyrazinecarbonitrile, PF-00477736, CCT241533, and SCH900776.

22. The method of claim 21, wherein the compound is 3-(2,4-dihydroxy-5-isopropyl-phenyl)-4-(1-methyl-indol-5-yl)-5-hydroxy-[1,2,4]triazole, or a tautomer or a pharmaceutically acceptable salt thereof and the CHK inhibitor is 5-(3-fluorophenyl)-3-ureidothiophene-N—[(S)-piperidin-3-yl]-2-carboxamide.

23. The method of claim 21, wherein the compound is 5-hydroxy-4-(5-hydroxy-4-(1-methyl-1H-indol-5-yl)-4H-1,2,4-triazol-3-yl)-2-isopropylphenyl dihydrogen phosphate, or a tautomer, or a pharmaceutically acceptable salt thereof, and the CHK inhibitor is 5-(3-fluorophenyl)-3-ureidothiophene-N—[(S)-piperidin-3-yl]-2-carboxamide.
